SEQUENCE LISTING (301 sequences)

SEQ ID NO: 1 - DNA, 20 bp, synthetic construct
  source 1..20: mol_type = other DNA
gctggtctatgtgatggtag

SEQ ID NO: 2 - DNA, 20 bp, synthetic construct
  source 1..20: mol_type = other DNA
atcagaggctgtatcagcac

SEQ ID NO: 3 - DNA, 20 bp, synthetic construct
  source 1..20: mol_type = other DNA
cctctaacagaagaaccatg

SEQ ID NO: 4 - DNA, 20 bp, synthetic construct
  source 1..20: mol_type = other DNA
tggttcattgggaataggtc

SEQ ID NO: 5 - DNA, 20 bp, synthetic construct
  source 1..20: mol_type = other DNA
caacctttgtgggcagagac

SEQ ID NO: 6 - DNA, 20 bp, synthetic construct
  source 1..20: mol_type = other DNA
ggtcatacagctcacagcag

SEQ ID NO: 7 - DNA, 20 bp, synthetic construct
  source 1..20: mol_type = other DNA
gcccaagtcagaaagtcaca

SEQ ID NO: 8 - DNA, 20 bp, synthetic construct
  source 1..20: mol_type = other DNA
agtgtagtgtgaggacaggg

SEQ ID NO: 9 - DNA, 20 bp, synthetic construct
  source 1..20: mol_type = other DNA
ttggactacacatgggggtc

SEQ ID NO: 10 - DNA, 20 bp, synthetic construct
  source 1..20: mol_type = other DNA
gcaaggaacaagaccacacc

SEQ ID NO: 11 - DNA, 20 bp, synthetic construct
  source 1..20: mol_type = other DNA
gcttatgaagagccaggtga

SEQ ID NO: 12 - DNA, 20 bp, synthetic construct
  source 1..20: mol_type = other DNA
gctcttcaggggactagatc

SEQ ID NO: 13 - DNA, 20 bp, synthetic construct
  source 1..20: mol_type = other DNA
ggtatagttggacaagctgt

SEQ ID NO: 14 - DNA, 20 bp, synthetic construct
  source 1..20: mol_type = other DNA
agacatatggacaggacctt

SEQ ID NO: 15 - DNA, 20 bp, synthetic construct
  source 1..20: mol_type = other DNA
ggcagctaacctagtgtaga

SEQ ID NO: 16 - DNA, 20 bp, synthetic construct
  source 1..20: mol_type = other DNA
tttcaccaacaggcttcatc

SEQ ID NO: 17 - DNA, 20 bp, synthetic construct
  source 1..20: mol_type = other DNA
caacaccctaccctaaacct

SEQ ID NO: 18 - DNA, 20 bp, synthetic construct
  source 1..20: mol_type = other DNA
ggaatctcaagggatacctg

SEQ ID NO: 19 - DNA, 20 bp, synthetic construct
  source 1..20: mol_type = other DNA
ggttaacttgaggggcagtc

SEQ ID NO: 20 - DNA, 20 bp, synthetic construct
  source 1..20: mol_type = other DNA
tagggcagggagaggaatac

SEQ ID NO: 21 - DNA, 20 bp, synthetic construct
  source 1..20: mol_type = other DNA
gcagtggccagaaggtttac

SEQ ID NO: 22 - DNA, 20 bp, synthetic construct
  source 1..20: mol_type = other DNA
gtagccaagatcagtgtgag

SEQ ID NO: 23 - DNA, 20 bp, synthetic construct
  source 1..20: mol_type = other DNA
gtggaagcaaaatatttaaa

SEQ ID NO: 24 - DNA, 20 bp, synthetic construct
  source 1..20: mol_type = other DNA
cagaaactatagaacaggct

SEQ ID NO: 25 - DNA, 20 bp, synthetic construct
  source 1..20: mol_type = other DNA
tctctggacccttagtatta

SEQ ID NO: 26 - DNA, 20 bp, synthetic construct
  source 1..20: mol_type = other DNA
gcaattgaaagtctctttgg

SEQ ID NO: 27 - DNA, 20 bp, synthetic construct
  source 1..20: mol_type = other DNA
gaattgagagtcttttgagc

SEQ ID NO: 28 - DNA, 20 bp, synthetic construct
  source 1..20: mol_type = other DNA
tggacaaagactgatgaccc

SEQ ID NO: 29 - DNA, 20 bp, synthetic construct
  source 1..20: mol_type = other DNA
agggtcatacagctcacagc

SEQ ID NO: 30 - DNA, 20 bp, synthetic construct
  source 1..20: mol_type = other DNA
gcttggctctatcttaagtg

SEQ ID NO: 31 - DNA, 20 bp, synthetic construct
  source 1..20: mol_type = other DNA
gtagtaggtgatgaagctgg

SEQ ID NO: 32 - DNA, 20 bp, synthetic construct
  source 1..20: mol_type = other DNA
gaggtcagtttggcttgtgt

SEQ ID NO: 33 - DNA, 20 bp, synthetic construct
  source 1..20: mol_type = other DNA
gttaacaaggcaaaggaggg

SEQ ID NO: 34 - DNA, 20 bp, synthetic construct
  source 1..20: mol_type = other DNA
ggctagtttttaacccagtt

SEQ ID NO: 35 - DNA, 20 bp, synthetic construct
  source 1..20: mol_type = other DNA
ggcaccataaacccagtatc

SEQ ID NO: 36 - DNA, 20 bp, synthetic construct
  source 1..20: mol_type = other DNA
taactcacagctgtttgaga

SEQ ID NO: 37 - DNA, 20 bp, synthetic construct
  source 1..20: mol_type = other DNA
ttgggagcttgattcagaca

SEQ ID NO: 38 - DNA, 20 bp, synthetic construct
  source 1..20: mol_type = other DNA
gatttagtgtagtgtgagga

SEQ ID NO: 39 - DNA, 20 bp, synthetic construct
  source 1..20: mol_type = other DNA
tcttagtgattggtgggtgg

SEQ ID NO: 40 - DNA, 20 bp, synthetic construct
  source 1..20: mol_type = other DNA
ggaccattagtagtatacct

SEQ ID NO: 41 - DNA, 20 bp, synthetic construct
  source 1..20: mol_type = other DNA
tctgagctcatggtatttgt

SEQ ID NO: 42 - DNA, 20 bp, synthetic construct
  source 1..20: mol_type = other DNA
ctcacctctcattggtcttc

SEQ ID NO: 43 - DNA, 20 bp, synthetic construct
  source 1..20: mol_type = other DNA
tacatggatggtcttcacta

SEQ ID NO: 44 - DNA, 20 bp, synthetic construct
  source 1..20: mol_type = other DNA
agggcatttgagctgacatt

SEQ ID NO: 45 - DNA, 20 bp, synthetic construct
  source 1..20: mol_type = other DNA
catagcaggcaacataagtg

SEQ ID NO: 46 - DNA, 20 bp, synthetic construct
  source 1..20: mol_type = other DNA
ttctttggactacacatggg

SEQ ID NO: 47 - DNA, 20 bp, synthetic construct
  source 1..20: mol_type = other DNA
tggccccctttaactggtta

SEQ ID NO: 48 - DNA, 20 bp, synthetic construct
  source 1..20: mol_type = other DNA
acaagaccacaccagccttt

SEQ ID NO: 49 - DNA, 20 bp, synthetic construct
  source 1..20: mol_type = other DNA
agcaaccagagcaaggaaca

SEQ ID NO: 50 - DNA, 20 bp, synthetic construct
  source 1..20: mol_type = other DNA
gttggagaaatgatagcagt

SEQ ID NO: 51 - DNA, 20 bp, synthetic construct
  source 1..20: mol_type = other DNA
gagccaggtgaagcccattc

SEQ ID NO: 52 - DNA, 20 bp, synthetic construct
  source 1..20: mol_type = other DNA
agcttctactctgggtcata

SEQ ID NO: 53 - DNA, 20 bp, synthetic construct
  source 1..20: mol_type = other DNA
gggcctattatgttgaactg

SEQ ID NO: 54 - DNA, 20 bp, synthetic construct
  source 1..20: mol_type = other DNA
ggttggaaacaaagcatggt

SEQ ID NO: 55 - DNA, 20 bp, synthetic construct
  source 1..20: mol_type = other DNA
gccagtacttctagccatac

SEQ ID NO: 56 - DNA, 20 bp, synthetic construct
  source 1..20: mol_type = other DNA
ctagtcaagctcttcagggg

SEQ ID NO: 57 - DNA, 20 bp, synthetic construct
  source 1..20: mol_type = other DNA
ggggtaaggaaacctaattg

SEQ ID NO: 58 - DNA, 20 bp, synthetic construct
  source 1..20: mol_type = other DNA
gcctaggctcagggggactt

SEQ ID NO: 59 - DNA, 20 bp, synthetic construct
  source 1..20: mol_type = other DNA
ggagtgattaatacaaaggg

SEQ ID NO: 60 - DNA, 20 bp, synthetic construct
  source 1..20: mol_type = other DNA
aacctagtgtagaagagtca

SEQ ID NO: 61 - DNA, 20 bp, synthetic construct
  source 1..20: mol_type = other DNA
caacccctcagcattattga

SEQ ID NO: 62 - DNA, 20 bp, synthetic construct
  source 1..20: mol_type = other DNA
tgtctctgggcatataaggg

SEQ ID NO: 63 - DNA, 20 bp, synthetic construct
  source 1..20: mol_type = other DNA
gattaagcttcaccatctac

SEQ ID NO: 64 - DNA, 20 bp, synthetic construct
  source 1..20: mol_type = other DNA
gtgttcacagagcatagata

SEQ ID NO: 65 - DNA, 20 bp, synthetic construct
  source 1..20: mol_type = other DNA
gaaggttttgagtactacac

SEQ ID NO: 66 - DNA, 20 bp, synthetic construct
  source 1..20: mol_type = other DNA
taggttaacttgaggggcag

SEQ ID NO: 67 - DNA, 20 bp, synthetic construct
  source 1..20: mol_type = other DNA
gcctgatgattttgaagact

SEQ ID NO: 68 - DNA, 20 bp, synthetic construct
  source 1..20: mol_type = other DNA
cagaaggtttactagaggac

SEQ ID NO: 69 - DNA, 20 bp, synthetic construct
  source 1..20: mol_type = other DNA
ggccaagtaaaatttgtgtg

SEQ ID NO: 70 - DNA, 20 bp, synthetic construct
  source 1..20: mol_type = other DNA
gtggcatgattcataggatt

SEQ ID NO: 71 - DNA, 20 bp, synthetic construct
  source 1..20: mol_type = other DNA
ggtctgactgtcttagtatc

SEQ ID NO: 72 - DNA, 20 bp, synthetic construct
  source 1..20: mol_type = other DNA
acagttgagcagagtttggc

SEQ ID NO: 73 - DNA, 20 bp, synthetic construct
  source 1..20: mol_type = other DNA
gagtgatcttagaaatacta

SEQ ID NO: 74 - DNA, 20 bp, synthetic construct
  source 1..20: mol_type = other DNA
gttatttgatcaaggttgtt

SEQ ID NO: 75 - DNA, 20 bp, synthetic construct
  source 1..20: mol_type = other DNA
tgtctctaagcaatctatac

SEQ ID NO: 76 - DNA, 20 bp, synthetic construct
  source 1..20: mol_type = other DNA
gggatgaaagttaattacaa

SEQ ID NO: 77 - DNA, 20 bp, synthetic construct
  source 1..20: mol_type = other DNA
catatatttctgtgatctag

SEQ ID NO: 78 - DNA, 20 bp, synthetic construct
  source 1..20: mol_type = other DNA
tacatcatcttacagtaaag

SEQ ID NO: 79 - DNA, 20 bp, synthetic construct
  source 1..20: mol_type = other DNA
ttgggcaatattctaaagtc

SEQ ID NO: 80 - DNA, 20 bp, synthetic construct
  source 1..20: mol_type = other DNA
ttacacaggccttaatatgg

SEQ ID NO: 81 - DNA, 20 bp, synthetic construct
  source 1..20: mol_type = other DNA
aaggaggggcaagaatgtac

SEQ ID NO: 82 - DNA, 20 bp, synthetic construct
  source 1..20: mol_type = other DNA
gtaaaattctagggtgtgaa

SEQ ID NO: 83 - DNA, 20 bp, synthetic construct
  source 1..20: mol_type = other DNA
gacatggacatcttttaatg

SEQ ID NO: 84 - DNA, 20 bp, synthetic construct
  source 1..20: mol_type = other DNA
aaaagatatggcttattggc

SEQ ID NO: 85 - DNA, 20 bp, synthetic construct
  source 1..20: mol_type = other DNA
gcatagcctgacacatacta

SEQ ID NO: 86 - DNA, 20 bp, synthetic construct
  source 1..20: mol_type = other DNA
ttacatctgtaattaggcca

SEQ ID NO: 87 - DNA, 20 bp, synthetic construct
  source 1..20: mol_type = other DNA
tagtggatttaaagtacagt

SEQ ID NO: 88 - DNA, 20 bp, synthetic construct
  source 1..20: mol_type = other DNA
ttgtatgagcctttcacata

SEQ ID NO: 89 - DNA, 20 bp, synthetic construct
  source 1..20: mol_type = other DNA
gcaagattgtacttaaatgg

SEQ ID NO: 90 - DNA, 20 bp, synthetic construct
  source 1..20: mol_type = other DNA
gtctgttacatatgtcatgt

SEQ ID NO: 91 - DNA, 20 bp, synthetic construct
  source 1..20: mol_type = other DNA
ctgatagaaaaagggcattt

SEQ ID NO: 92 - DNA, 20 bp, synthetic construct
  source 1..20: mol_type = other DNA
acaagaattatacttgtagc

SEQ ID NO: 93 - DNA, 20 bp, synthetic construct
  source 1..20: mol_type = other DNA
ccatgactgaaattcacagt

SEQ ID NO: 94 - DNA, 20 bp, synthetic construct
  source 1..20: mol_type = other DNA
aatatggttcatttcagcca

SEQ ID NO: 95 - DNA, 20 bp, synthetic construct
  source 1..20: mol_type = other DNA
tgagatagggagtaaaacaa

SEQ ID NO: 96 - DNA, 20 bp, synthetic construct
  source 1..20: mol_type = other DNA
acattgagataagtgtcatg

SEQ ID NO: 97 - DNA, 20 bp, synthetic construct
  source 1..20: mol_type = other DNA
ttataagctagtggggaata

SEQ ID NO: 98 - DNA, 20 bp, synthetic construct
  source 1..20: mol_type = other DNA
atacaagacaagcttataca

SEQ ID NO: 99 - DNA, 20 bp, synthetic construct
  source 1..20: mol_type = other DNA
tgaactcatatcatctgtgg

SEQ ID NO: 100 - DNA, 20 bp, synthetic construct
  source 1..20: mol_type = other DNA
gcttagagctcatatggaat

SEQ ID NO: 101 - DNA, 20 bp, synthetic construct
  source 1..20: mol_type = other DNA
ataggaacttatgagaattg

SEQ ID NO: 102 - DNA, 20 bp, synthetic construct
  source 1..20: mol_type = other DNA
gttaaatcaagaactaagtc

SEQ ID NO: 103 - DNA, 20 bp, synthetic construct
  source 1..20: mol_type = other DNA
aggtactaattgttacatgg

SEQ ID NO: 104 - DNA, 20 bp, synthetic construct
  source 1..20: mol_type = other DNA
acttgtaaatagatctagtc

SEQ ID NO: 105 - DNA, 20 bp, synthetic construct
  source 1..20: mol_type = other DNA
ttaactctggcctagtgtac

SEQ ID NO: 106 - DNA, 20 bp, synthetic construct
  source 1..20: mol_type = other DNA
cctgttggtacactaaattt

SEQ ID NO: 107 - DNA, 20 bp, synthetic construct
  source 1..20: mol_type = other DNA
ccagatgattatagttgaaa

SEQ ID NO: 108 - DNA, 20 bp, synthetic construct
  source 1..20: mol_type = other DNA
tatggccattaatctgtaac

SEQ ID NO: 109 - DNA, 20 bp, synthetic construct
  source 1..20: mol_type = other DNA
ctagctaatgggtaatgtca

SEQ ID NO: 110 - DNA, 20 bp, synthetic construct
  source 1..20: mol_type = other DNA
acagctattgtctaccaact

SEQ ID NO: 111 - DNA, 20 bp, synthetic construct
  source 1..20: mol_type = other DNA
atattctaaaatagggaagc

SEQ ID NO: 112 - DNA, 20 bp, synthetic construct
  source 1..20: mol_type = other DNA
ctctatgatgagaattcaag

SEQ ID NO: 113 - DNA, 20 bp, synthetic construct
  source 1..20: mol_type = other DNA
acccaacttaacagagacat

SEQ ID NO: 114 - DNA, 20 bp, synthetic construct
  source 1..20: mol_type = other DNA
gaatattgattaacccaact

SEQ ID NO: 115 - DNA, 20 bp, synthetic construct
  source 1..20: mol_type = other DNA
aatctggttaatgagttagg

SEQ ID NO: 116 - DNA, 20 bp, synthetic construct
  source 1..20: mol_type = other DNA
cataattttagtcagtgtgg

SEQ ID NO: 117 - DNA, 20 bp, synthetic construct
  source 1..20: mol_type = other DNA
ttatcaatgaggactgattt

SEQ ID NO: 118 - DNA, 20 bp, synthetic construct
  source 1..20: mol_type = other DNA
caaaccctgtgagtaaattc

SEQ ID NO: 119 - DNA, 20 bp, synthetic construct
  source 1..20: mol_type = other DNA
gtctgttttgatgagatggt

SEQ ID NO: 120 - DNA, 20 bp, synthetic construct
  source 1..20: mol_type = other DNA
tatatgtctaagactaggtg

SEQ ID NO: 121 - DNA, 20 bp, synthetic construct
  source 1..20: mol_type = other DNA
ttaacccagtttgtgtttag

SEQ ID NO: 122 - DNA, 20 bp, synthetic construct
  source 1..20: mol_type = other DNA
ttttaacccagtttgtgttt

SEQ ID NO: 123 - DNA, 20 bp, synthetic construct
  source 1..20: mol_type = other DNA
gtttttaacccagtttgtgt

SEQ ID NO: 124 - DNA, 20 bp, synthetic construct
  source 1..20: mol_type = other DNA
tagtttttaacccagtttgt

SEQ ID NO: 125 - DNA, 20 bp, synthetic construct
  source 1..20: mol_type = other DNA
ttggctagtttttaacccag

SEQ ID NO: 126 - DNA, 20 bp, synthetic construct
  source 1..20: mol_type = other DNA
tatttggctagtttttaacc

SEQ ID NO: 127 - DNA, 20 bp, synthetic construct
  source 1..20: mol_type = other DNA
ctatttggctagtttttaac

SEQ ID NO: 128 - DNA, 20 bp, synthetic construct
  source 1..20: mol_type = other DNA
tactatttggctagttttta

SEQ ID NO: 129 - DNA, 20 bp, synthetic construct
  source 1..20: mol_type = other DNA
ataagggaaatgattcagac

SEQ ID NO: 130 - DNA, 20 bp, synthetic construct
  source 1..20: mol_type = other DNA
gcatataagggaaatgattc

SEQ ID NO: 131 - DNA, 20 bp, synthetic construct
  source 1..20: mol_type = other DNA
gggcatataagggaaatgat

SEQ ID NO: 132 - DNA, 20 bp, synthetic construct
  source 1..20: mol_type = other DNA
ctgggcatataagggaaatg

SEQ ID NO: 133 - DNA, 20 bp, synthetic construct
  source 1..20: mol_type = other DNA
tctctgggcatataagggaa

SEQ ID NO: 134 - DNA, 20 bp, synthetic construct
  source 1..20: mol_type = other DNA
gctgtctctgggcatataag

SEQ ID NO: 135 - DNA, 20 bp, synthetic construct
  source 1..20: mol_type = other DNA
tatgctgtctctgggcatat

SEQ ID NO: 136 - DNA, 20 bp, synthetic construct
  source 1..20: mol_type = other DNA
actatgctgtctctgggcat

SEQ ID NO: 137 - DNA, 20 bp, synthetic construct
  source 1..20: mol_type = other DNA
aagtaaaatttgtgtgtgca

SEQ ID NO: 138 - DNA, 20 bp, synthetic construct
  source 1..20: mol_type = other DNA
ccaagtaaaatttgtgtgtg

SEQ ID NO: 139 - DNA, 20 bp, synthetic construct
  source 1..20: mol_type = other DNA
gtggccaagtaaaatttgtg

SEQ ID NO: 140 - DNA, 20 bp, synthetic construct
  source 1..20: mol_type = other DNA
cctgtggccaagtaaaattt

SEQ ID NO: 141 - DNA, 20 bp, synthetic construct
  source 1..20: mol_type = other DNA
catcctgtggccaagtaaaa

SEQ ID NO: 142 - DNA, 20 bp, synthetic construct
  source 1..20: mol_type = other DNA
ttacatcctgtggccaagta

SEQ ID NO: 143 - DNA, 20 bp, synthetic construct
  source 1..20: mol_type = other DNA
atttacatcctgtggccaag

SEQ ID NO: 144 - DNA, 20 bp, synthetic construct
  source 1..20: mol_type = other DNA
cccatttacatcctgtggcc

SEQ ID NO: 145 - DNA, 20 bp, synthetic construct
  source 1..20: mol_type = other DNA
actatgaaagttaagacagt

SEQ ID NO: 146 - DNA, 20 bp, synthetic construct
  source 1..20: mol_type = other DNA
tacttactatgaaagttaag

SEQ ID NO: 147 - DNA, 20 bp, synthetic construct
  source 1..20: mol_type = other DNA
cttaccaagtaattatagtg

SEQ ID NO: 148 - DNA, 20 bp, synthetic construct
  source 1..20: mol_type = other DNA
catataccttcagcccaaag

SEQ ID NO: 149 - DNA, 20 bp, synthetic construct
  source 1..20: mol_type = other DNA
gcaacacatataccttcagc

SEQ ID NO: 150 - DNA, 20 bp, synthetic construct
  source 1..20: mol_type = other DNA
gtatcagcaacacatatacc

SEQ ID NO: 151 - DNA, 20 bp, Homo sapiens
  source 1..20: mol_type = genomic DNA
ctaccatcacatagaccagc

SEQ ID NO: 152 - DNA, 20 bp, Homo sapiens
  source 1..20: mol_type = genomic DNA
gtgctgatacagcctctgat

SEQ ID NO: 153 - DNA, 20 bp, Homo sapiens
  source 1..20: mol_type = genomic DNA
catggttcttctgttagagg

SEQ ID NO: 154 - DNA, 20 bp, Homo sapiens
  source 1..20: mol_type = genomic DNA
gacctattcccaatgaacca

SEQ ID NO: 155 - DNA, 20 bp, Homo sapiens
  source 1..20: mol_type = genomic DNA
gtctctgcccacaaaggttg

SEQ ID NO: 156 - DNA, 20 bp, Homo sapiens
  source 1..20: mol_type = genomic DNA
ctgctgtgagctgtatgacc

SEQ ID NO: 157 - DNA, 20 bp, Homo sapiens
  source 1..20: mol_type = genomic DNA
tgtgactttctgacttgggc

SEQ ID NO: 158 - DNA, 20 bp, Homo sapiens
  source 1..20: mol_type = genomic DNA
ccctgtcctcacactacact

SEQ ID NO: 159 - DNA, 20 bp, Homo sapiens
  source 1..20: mol_type = genomic DNA
gacccccatgtgtagtccaa

SEQ ID NO: 160 - DNA, 20 bp, Homo sapiens
  source 1..20: mol_type = genomic DNA
ggtgtggtcttgttccttgc

SEQ ID NO: 161 - DNA, 20 bp, Homo sapiens
  source 1..20: mol_type = genomic DNA
tcacctggctcttcataagc

SEQ ID NO: 162 - DNA, 20 bp, Homo sapiens
  source 1..20: mol_type = genomic DNA
gatctagtcccctgaagagc

SEQ ID NO: 163 - DNA, 20 bp, Homo sapiens
  source 1..20: mol_type = genomic DNA
acagcttgtccaactatacc

SEQ ID NO: 164 - DNA, 20 bp, Homo sapiens
  source 1..20: mol_type = genomic DNA
aaggtcctgtccatatgtct

SEQ ID NO: 165 - DNA, 20 bp, Homo sapiens
  source 1..20: mol_type = genomic DNA
tctacactaggttagctgcc

SEQ ID NO: 166 - DNA, 20 bp, Homo sapiens
  source 1..20: mol_type = genomic DNA
gatgaagcctgttggtgaaa

SEQ ID NO: 167 - DNA, 20 bp, Homo sapiens
  source 1..20: mol_type = genomic DNA
aggtttagggtagggtgttg

SEQ ID NO: 168 - DNA, 20 bp, Homo sapiens
  source 1..20: mol_type = genomic DNA
caggtatcccttgagattcc

SEQ ID NO: 169 - DNA, 20 bp, Homo sapiens
  source 1..20: mol_type = genomic DNA
gactgcccctcaagttaacc

SEQ ID NO: 170 - DNA, 20 bp, Homo sapiens
  source 1..20: mol_type = genomic DNA
gtattcctctccctgcccta

SEQ ID NO: 171 - DNA, 20 bp, Homo sapiens
  source 1..20: mol_type = genomic DNA
gtaaaccttctggccactgc

SEQ ID NO: 172 - DNA, 20 bp, Homo sapiens
  source 1..20: mol_type = genomic DNA
ctcacactgatcttggctac

SEQ ID NO: 173 - DNA, 20 bp, Homo sapiens
  source 1..20: mol_type = genomic DNA
tttaaatattttgcttccac

SEQ ID NO: 174 - DNA, 20 bp, Homo sapiens
  source 1..20: mol_type = genomic DNA
agcctgttctatagtttctg

SEQ ID NO: 175 - DNA, 20 bp, Homo sapiens
  source 1..20: mol_type = genomic DNA
taatactaagggtccagaga

SEQ ID NO: 176 - DNA, 20 bp, Homo sapiens
  source 1..20: mol_type = genomic DNA
ccaaagagactttcaattgc

SEQ ID NO: 177 - DNA, 20 bp, Homo sapiens
  source 1..20: mol_type = genomic DNA
gctcaaaagactctcaattc

SEQ ID NO: 178 - DNA, 20 bp, Homo sapiens
  source 1..20: mol_type = genomic DNA
gggtcatcagtctttgtcca

SEQ ID NO: 179 - DNA, 20 bp, Homo sapiens
  source 1..20: mol_type = genomic DNA
gctgtgagctgtatgaccct

SEQ ID NO: 180 - DNA, 20 bp, Homo sapiens
  source 1..20: mol_type = genomic DNA
cacttaagatagagccaagc

SEQ ID NO: 181 - DNA, 20 bp, Homo sapiens
  source 1..20: mol_type = genomic DNA
ccagcttcatcacctactac

SEQ ID NO: 182 - DNA, 20 bp, Homo sapiens
  source 1..20: mol_type = genomic DNA
acacaagccaaactgacctc

SEQ ID NO: 183 - DNA, 20 bp, Homo sapiens
  source 1..20: mol_type = genomic DNA
ccctcctttgccttgttaac

SEQ ID NO: 184 - DNA, 20 bp, Homo sapiens
  source 1..20: mol_type = genomic DNA
aactgggttaaaaactagcc

SEQ ID NO: 185 - DNA, 20 bp, Homo sapiens
  source 1..20: mol_type = genomic DNA
gatactgggtttatggtgcc

SEQ ID NO: 186 - DNA, 20 bp, Homo sapiens
  source 1..20: mol_type = genomic DNA
tctcaaacagctgtgagtta

SEQ ID NO: 187 - DNA, 20 bp, Homo sapiens
  source 1..20: mol_type = genomic DNA
tgtctgaatcaagctcccaa

SEQ ID NO: 188 - DNA, 20 bp, Homo sapiens
  source 1..20: mol_type = genomic DNA
tcctcacactacactaaatc

SEQ ID NO: 189 - DNA, 20 bp, Homo sapiens
  source 1..20: mol_type = genomic DNA
ccacccaccaatcactaaga

SEQ ID NO: 190 - DNA, 20 bp, Homo sapiens
  source 1..20: mol_type = genomic DNA
aggtatactactaatggtcc

SEQ ID NO: 191 - DNA, 20 bp, Homo sapiens
  source 1..20: mol_type = genomic DNA
acaaataccatgagctcaga

SEQ ID NO: 192 - DNA, 20 bp, Homo sapiens
  source 1..20: mol_type = genomic DNA
gaagaccaatgagaggtgag

SEQ ID NO: 193 - DNA, 20 bp, Homo sapiens
  source 1..20: mol_type = genomic DNA
tagtgaagaccatccatgta

SEQ ID NO: 194 - DNA, 20 bp, Homo sapiens
  source 1..20: mol_type = genomic DNA
aatgtcagctcaaatgccct

SEQ ID NO: 195 - DNA, 20 bp, Homo sapiens
  source 1..20: mol_type = genomic DNA
cacttatgttgcctgctatg

SEQ ID NO: 196 - DNA, 20 bp, Homo sapiens
  source 1..20: mol_type = genomic DNA
cccatgtgtagtccaaagaa

SEQ ID NO: 197 - DNA, 20 bp, Homo sapiens
  source 1..20: mol_type = genomic DNA
taaccagttaaagggggcca

SEQ ID NO: 198 - DNA, 20 bp, Homo sapiens
  source 1..20: mol_type = genomic DNA
aaaggctggtgtggtcttgt

SEQ ID NO: 199 - DNA, 20 bp, Homo sapiens
  source 1..20: mol_type = genomic DNA
tgttccttgctctggttgct

SEQ ID NO: 200 - DNA, 20 bp, Homo sapiens
  source 1..20: mol_type = genomic DNA
actgctatcatttctccaac

SEQ ID NO: 201 - DNA, 20 bp, Homo sapiens
  source 1..20: mol_type = genomic DNA
gaatgggcttcacctggctc

SEQ ID NO: 202 - DNA, 20 bp, Homo sapiens
  source 1..20: mol_type = genomic DNA
tatgacccagagtagaagct

SEQ ID NO: 203 - DNA, 20 bp, Homo sapiens
  source 1..20: mol_type = genomic DNA
cagttcaacataataggccc

SEQ ID NO: 204 - DNA, 20 bp, Homo sapiens
  source 1..20: mol_type = genomic DNA
accatgctttgtttccaacc

SEQ ID NO: 205 - DNA, 20 bp, Homo sapiens
  source 1..20: mol_type = genomic DNA
gtatggctagaagtactggc

SEQ ID NO: 206 - DNA, 20 bp, Homo sapiens
  source 1..20: mol_type = genomic DNA
cccctgaagagcttgactag

SEQ ID NO: 207 - DNA, 20 bp, Homo sapiens
  source 1..20: mol_type = genomic DNA
caattaggtttccttacccc

SEQ ID NO: 208 - DNA, 20 bp, Homo sapiens
  source 1..20: mol_type = genomic DNA
aagtccccctgagcctaggc

SEQ ID NO: 209 - DNA, 20 bp, Homo sapiens
  source 1..20: mol_type = genomic DNA
ccctttgtattaatcactcc

SEQ ID NO: 210 - DNA, 20 bp, Homo sapiens
  source 1..20: mol_type = genomic DNA
tgactcttctacactaggtt

SEQ ID NO: 211 - DNA, 20 bp, Homo sapiens
  source 1..20: mol_type = genomic DNA
tcaataatgctgaggggttg

SEQ ID NO: 212 - DNA, 20 bp, Homo sapiens
  source 1..20: mol_type = genomic DNA
cccttatatgcccagagaca

SEQ ID NO: 213 - DNA, 20 bp, Homo sapiens
  source 1..20: mol_type = genomic DNA
gtagatggtgaagcttaatc

SEQ ID NO: 214 - DNA, 20 bp, Homo sapiens
  source 1..20: mol_type = genomic DNA
tatctatgctctgtgaacac

SEQ ID NO: 215 - DNA, 20 bp, Homo sapiens
  source 1..20: mol_type = genomic DNA
gtgtagtactcaaaaccttc

SEQ ID NO: 216 - DNA, 20 bp, Homo sapiens
  source 1..20: mol_type = genomic DNA
ctgcccctcaagttaaccta

SEQ ID NO: 217 - DNA, 20 bp, Homo sapiens
  source 1..20: mol_type = genomic DNA
agtcttcaaaatcatcaggc

SEQ ID NO: 218 - DNA, 20 bp, Homo sapiens
  source 1..20: mol_type = genomic DNA
gtcctctagtaaaccttctg

SEQ ID NO: 219 - DNA, 20 bp, Homo sapiens
  source 1..20: mol_type = genomic DNA
cacacaaattttacttggcc

SEQ ID NO: 220 - DNA, 20 bp, Homo sapiens
  source 1..20: mol_type = genomic DNA
aatcctatgaatcatgccac

SEQ ID NO: 221 - DNA, 20 bp, Homo sapiens
  source 1..20: mol_type = genomic DNA
gatactaagacagtcagacc

SEQ ID NO: 222 - DNA, 20 bp, Homo sapiens
  source 1..20: mol_type = genomic DNA
gccaaactctgctcaactgt

SEQ ID NO: 223 - DNA, 20 bp, Homo sapiens
  source 1..20: mol_type = genomic DNA
tagtatttctaagatcactc

SEQ ID NO: 224 - DNA, 20 bp, Homo sapiens
  source 1..20: mol_type = genomic DNA
aacaaccttgatcaaataac

SEQ ID NO: 225 - DNA, 20 bp, Homo sapiens
  source 1..20: mol_type = genomic DNA
gtatagattgcttagagaca

SEQ ID NO: 226 - DNA, 20 bp, Homo sapiens
  source 1..20: mol_type = genomic DNA
ttgtaattaactttcatccc

SEQ ID NO: 227 - DNA, 20 bp, Homo sapiens
  source 1..20: mol_type = genomic DNA
ctagatcacagaaatatatg

SEQ ID NO: 228 - DNA, 20 bp, Homo sapiens
  source 1..20: mol_type = genomic DNA
ctttactgtaagatgatgta

SEQ ID NO: 229 - DNA, 20 bp, Homo sapiens
  source 1..20: mol_type = genomic DNA
gactttagaatattgcccaa

SEQ ID NO: 230 - DNA, 20 bp, Homo sapiens
  source 1..20: mol_type = genomic DNA
ccatattaaggcctgtgtaa

SEQ ID NO: 231 - DNA, 20 bp, Homo sapiens
  source 1..20: mol_type = genomic DNA
gtacattcttgcccctcctt

SEQ ID NO: 232 - DNA, 20 bp, Homo sapiens
  source 1..20: mol_type = genomic DNA
ttcacaccctagaattttac

SEQ ID NO: 233 - DNA, 20 bp, Homo sapiens
  source 1..20: mol_type = genomic DNA
cattaaaagatgtccatgtc

SEQ ID NO: 234 - DNA, 20 bp, Homo sapiens
  source 1..20: mol_type = genomic DNA
gccaataagccatatctttt

SEQ ID NO: 235 - DNA, 20 bp, Homo sapiens
  source 1..20: mol_type = genomic DNA
tagtatgtgtcaggctatgc

SEQ ID NO: 236 - DNA, 20 bp, Homo sapiens
  source 1..20: mol_type = genomic DNA
tggcctaattacagatgtaa

SEQ ID NO: 237 - DNA, 20 bp, Homo sapiens
  source 1..20: mol_type = genomic DNA
actgtactttaaatccacta

SEQ ID NO: 238 - DNA, 20 bp, Homo sapiens
  source 1..20: mol_type = genomic DNA
tatgtgaaaggctcatacaa

SEQ ID NO: 239 - DNA, 20 bp, Homo sapiens
  source 1..20: mol_type = genomic DNA
ccatttaagtacaatcttgc

SEQ ID NO: 240 - DNA, 20 bp, Homo sapiens
  source 1..20: mol_type = genomic DNA
acatgacatatgtaacagac

SEQ ID NO: 241 - DNA, 20 bp, Homo sapiens
  source 1..20: mol_type = genomic DNA
aaatgccctttttctatcag

SEQ ID NO: 242 - DNA, 20 bp, Homo sapiens
  source 1..20: mol_type = genomic DNA
gctacaagtataattcttgt

SEQ ID NO: 243 - DNA, 20 bp, Homo sapiens
  source 1..20: mol_type = genomic DNA
actgtgaatttcagtcatgg

SEQ ID NO: 244 - DNA, 20 bp, Homo sapiens
  source 1..20: mol_type = genomic DNA
tggctgaaatgaaccatatt

SEQ ID NO: 245 - DNA, 20 bp, Homo sapiens
  source 1..20: mol_type = genomic DNA
ttgttttactccctatctca

SEQ ID NO: 246 - DNA, 20 bp, Homo sapiens
  source 1..20: mol_type = genomic DNA
catgacacttatctcaatgt

SEQ ID NO: 247 - DNA, 20 bp, Homo sapiens
  source 1..20: mol_type = genomic DNA
tattccccactagcttataa

SEQ ID NO: 248 - DNA, 20 bp, Homo sapiens
  source 1..20: mol_type = genomic DNA
tgtataagcttgtcttgtat

SEQ ID NO: 249 - DNA, 20 bp, Homo sapiens
  source 1..20: mol_type = genomic DNA
ccacagatgatatgagttca

SEQ ID NO: 250 - DNA, 20 bp, Homo sapiens
  source 1..20: mol_type = genomic DNA
attccatatgagctctaagc

SEQ ID NO: 251 - DNA, 20 bp, Homo sapiens
  source 1..20: mol_type = genomic DNA
caattctcataagttcctat

SEQ ID NO: 252 - DNA, 20 bp, Homo sapiens
  source 1..20: mol_type = genomic DNA
gacttagttcttgatttaac

SEQ ID NO: 253 - DNA, 20 bp, Homo sapiens
  source 1..20: mol_type = genomic DNA
ccatgtaacaattagtacct

SEQ ID NO: 254 - DNA, 20 bp, Homo sapiens
  source 1..20: mol_type = genomic DNA
gactagatctatttacaagt

SEQ ID NO: 255 - DNA, 20 bp, Homo sapiens
  source 1..20: mol_type = genomic DNA
gtacactaggccagagttaa

SEQ ID NO: 256 - DNA, 20 bp, Homo sapiens
  source 1..20: mol_type = genomic DNA
aaatttagtgtaccaacagg

SEQ ID NO: 257 - DNA, 20 bp, Homo sapiens
  source 1..20: mol_type = genomic DNA
tttcaactataatcatctgg

SEQ ID NO: 258 - DNA, 20 bp, Homo sapiens
  source 1..20: mol_type = genomic DNA
gttacagattaatggccata

SEQ ID NO: 259 - DNA, 20 bp, Homo sapiens
  source 1..20: mol_type = genomic DNA
tgacattacccattagctag

SEQ ID NO: 260 - DNA, 20 bp, Homo sapiens
  source 1..20: mol_type = genomic DNA
agttggtagacaatagctgt

SEQ ID NO: 261 - DNA, 20 bp, Homo sapiens
  source 1..20: mol_type = genomic DNA
gcttccctattttagaatat

SEQ ID NO: 262 - DNA, 20 bp, Homo sapiens
  source 1..20: mol_type = genomic DNA
cttgaattctcatcatagag

SEQ ID NO: 263 - DNA, 20 bp, Homo sapiens
  source 1..20: mol_type = genomic DNA
atgtctctgttaagttgggt

SEQ ID NO: 264 - DNA, 20 bp, Homo sapiens
  source 1..20: mol_type = genomic DNA
agttgggttaatcaatattc

SEQ ID NO: 265 - DNA, 20 bp, Homo sapiens
  source 1..20: mol_type = genomic DNA
cctaactcattaaccagatt

SEQ ID NO: 266 - DNA, 20 bp, Homo sapiens
  source 1..20: mol_type = genomic DNA
ccacactgactaaaattatg

SEQ ID NO: 267 - DNA, 20 bp, Homo sapiens
  source 1..20: mol_type = genomic DNA
aaatcagtcctcattgataa

SEQ ID NO: 268 - DNA, 20 bp, Homo sapiens
  source 1..20: mol_type = genomic DNA
gaatttactcacagggtttg

SEQ ID NO: 269 - DNA, 20 bp, Homo sapiens
  source 1..20: mol_type = genomic DNA
accatctcatcaaaacagac

SEQ ID NO: 270 - DNA, 20 bp, Homo sapiens
  source 1..20: mol_type = genomic DNA
cacctagtcttagacatata

SEQ ID NO: 271 - DNA, 20 bp, Homo sapiens
  source 1..20: mol_type = genomic DNA
ctaaacacaaactgggttaa

SEQ ID NO: 272 - DNA, 20 bp, Homo sapiens
  source 1..20: mol_type = genomic DNA
aaacacaaactgggttaaaa

SEQ ID NO: 273 - DNA, 20 bp, Homo sapiens
  source 1..20: mol_type = genomic DNA
acacaaactgggttaaaaac

SEQ ID NO: 274 - DNA, 20 bp, Homo sapiens
  source 1..20: mol_type = genomic DNA
acaaactgggttaaaaacta

SEQ ID NO: 275 - DNA, 20 bp, Homo sapiens
  source 1..20: mol_type = genomic DNA
tgggttaaaaactagccaaa

SEQ ID NO: 276 - DNA, 20 bp, Homo sapiens
  source 1..20: mol_type = genomic DNA
ggttaaaaactagccaaata

SEQ ID NO: 277 - DNA, 20 bp, Homo sapiens
  source 1..20: mol_type = genomic DNA
gttaaaaactagccaaatag

SEQ ID NO: 278 - DNA, 20 bp, Homo sapiens
  source 1..20: mol_type = genomic DNA
taaaaactagccaaatagta

SEQ ID NO: 279 - DNA, 20 bp, Homo sapiens
  source 1..20: mol_type = genomic DNA
gtctgaatcatttcccttat

SEQ ID NO: 280 - DNA, 20 bp, Homo sapiens
  source 1..20: mol_type = genomic DNA
gaatcatttcccttatatgc

SEQ ID NO: 281 - DNA, 20 bp, Homo sapiens
  source 1..20: mol_type = genomic DNA
atcatttcccttatatgccc

SEQ ID NO: 282 - DNA, 20 bp, Homo sapiens
  source 1..20: mol_type = genomic DNA
catttcccttatatgcccag

SEQ ID NO: 283 - DNA, 20 bp, Homo sapiens
  source 1..20: mol_type = genomic DNA
ttcccttatatgcccagaga

SEQ ID NO: 284 - DNA, 20 bp, Homo sapiens
  source 1..20: mol_type = genomic DNA
cttatatgcccagagacagc

SEQ ID NO: 285 - DNA, 20 bp, Homo sapiens
  source 1..20: mol_type = genomic DNA
atatgcccagagacagcata

SEQ ID NO: 286 - DNA, 20 bp, Homo sapiens
  source 1..20: mol_type = genomic DNA
atgcccagagacagcatagt

SEQ ID NO: 287 - DNA, 20 bp, Homo sapiens
  source 1..20: mol_type = genomic DNA
tgcacacacaaattttactt

SEQ ID NO: 288 - DNA, 20 bp, Homo sapiens
  source 1..20: mol_type = genomic DNA
cacacacaaattttacttgg

SEQ ID NO: 289 - DNA, 20 bp, Homo sapiens
  source 1..20: mol_type = genomic DNA
cacaaattttacttggccac

SEQ ID NO: 290 - DNA, 20 bp, Homo sapiens
  source 1..20: mol_type = genomic DNA
aaattttacttggccacagg

SEQ ID NO: 291 - DNA, 20 bp, Homo sapiens
  source 1..20: mol_type = genomic DNA
ttttacttggccacaggatg

SEQ ID NO: 292 - DNA, 20 bp, Homo sapiens
  source 1..20: mol_type = genomic DNA
tacttggccacaggatgtaa

SEQ ID NO: 293 - DNA, 20 bp, Homo sapiens
  source 1..20: mol_type = genomic DNA
cttggccacaggatgtaaat

SEQ ID NO: 294 - DNA, 20 bp, Homo sapiens
  source 1..20: mol_type = genomic DNA
ggccacaggatgtaaatggg

SEQ ID NO: 295 - DNA, 20 bp, Homo sapiens
  source 1..20: mol_type = genomic DNA
actgtcttaactttcatagt

SEQ ID NO: 296 - DNA, 20 bp, Homo sapiens
  source 1..20: mol_type = genomic DNA
cttaactttcatagtaagta

SEQ ID NO: 297 - DNA, 20 bp, Homo sapiens
  source 1..20: mol_type = genomic DNA
cactataattacttggtaag

SEQ ID NO: 298 - DNA, 20 bp, Homo sapiens
  source 1..20: mol_type = genomic DNA
ctttgggctgaaggtatatg

SEQ ID NO: 299 - DNA, 20 bp, Homo sapiens
  source 1..20: mol_type = genomic DNA
gctgaaggtatatgtgttgc

SEQ ID NO: 300 - DNA, 20 bp, Homo sapiens
  source 1..20: mol_type = genomic DNA
ggtatatgtgttgctgatac

SEQ ID NO: 301 - DNA, 32306 bp, Homo sapiens
  source 1..32306: mol_type = genomic DNA
gccaatatgagtgatcatggtcttgggaacagtcttaagaggttctgaggaagtatgcctgaagcaggtaggttatagtttggttttatacatttccaggagaaaagagttacaggcgaagacataaatcgatacatggaaagtatatattgattcagcccaaaaaagcaggacatctccaagcagggacttagtagttataggtgggtcttaaggattcttcagttgacaattggttgagagggttaagctattgtctaaaggcttaaagtcaatagtaagtaatgcttgagttaaaggaggttgtggggtccaaggttcttgttacgttgatgaatcctcataggtagcagccctccgagagaatcaatggcaaacgtctgtctctttgcagacctttgagatgtcagactctcagttcaactctgccagaactgggaaaagcctagaaagggaaggcctggttgcattaatggagattatctacagatgcaaatctcactcacaaaagatagctttgcagggccatttcaaaatatgtcaaataaatatattttgaggtaaaatatttttattccctacagggtctgctatctgtcatgtgatgctatatcagagtcaggttgaaaagcaaaccatattacagcaggtttatttttatttatttatttattttgaggcagcgttttgctctcgttgcccaggctggagtgcaatggcacgatcttggctcactgcaacctctgcctcccaggttcaagcaattctgctgcctcagcctcctgagtagctgggattacaggcacctgtcaccacacccagctaatttttgtatttttagtagagatgaggtttcaccatattggccagactggtcttgaactcctgacctcaggtgatctgcctgcctcagcctcccaaagtgctgggattacaggagtgagccaccacgcccggactataccaggttaacttttaaaactcatttaatgatattttatggtttgtagggtgtgattccttgcatgaatggcctgaggtcttgtttataatttggtatcttattgccacagagtctgttttgtccgtcttatgatattttaacttgaatgctggtcagttgtgcctaaactccaaagggagggggtataacaaggcatgtctaacctcccttcccatcacggccaggaattcaatttttaagatgtttctggggtctctttggccaagtgggagttcattcagtcaatagggggcaagttgggggtgcttagcattttatgtttagtctacacactttagtccttgtgtcctgactgccactcaaatagactcaggacctcagaccactttagttagccagtggtgaagctagccatgacctgggtttctccctctggagcacagaattctcctctgtcccaaggcctgcctaaatgcactgtcagaattctaggcgcctgcagaattctgtcctgtgttatgttccaccatgacggggcagcactgacttccagtgcaaagtcccacactcactttgctctctctcctccaagcacacggattctctttcctgctgtgcagcctgtggttggaggaggggtggtataggcaatacagggagatcttcccccctacccccaccctatcttcaatgcctctttccttatcattacggtaaaagtaggcactgtgatcactcacctgattttatggcttttatgaaggtgctgtcttgcgtggatagttgttctatttggtgttcctgtgtggagaactatctcggggggggttctattcagccatcttgctccacctccgccctctctccagtcagtctttaaactgctggcaccgaagcctccagtgcctttctcctctatatcccatggagagttactgaagtagttctttttggatttcagttggccttttagtagagcctttctcctaaaggattaaaacgtgagactgcgggcttgagccaaaaagcagtcagagggacaaatactgggttttacttagaataacccacctgcctagtgccagcctaccactcttgaacaaaacttgtatgattcccgtgtctccatttagaataatttaattctgaggagacgcctctgtttctccactggcctctggacatggaacaggcacatgaaaattacctggggcagctatgggtcacatcacctctcttactctagccagccagcaggcacttgaagttcgcaatctgatgggctgaatttctgaaaacaataacaaacacaatgtcacttattttttattctctaaatttacaacccattaatatacctccctgtaagctgtctgattttgaaaattatacatgataaaatctttttattatacataaaaaagagttctgtagtttgtattaatagaaaattggtgtccagacatattttaccaagtaagtacaaaataactaaagaatatccatttgaaagatacaaaattattatcctgctaggagcaccttggtgtcaaggtgacttcagggaataatcaggtaattcagctccttggccggagtgaagaatggaatgttgaggctggagaactaagcagggctcaagaggggcctggtgcactaggctaggtcatttgggctcaatcatgaggggacaattgaggcagcattagcaggaaggtcaggtgaccaccaaactttctgccagatctgagtttcttctcttttagaaatgcagtatttgtataaaaatgaaaggcttaccaaaggtcacattttaaattataaagaaaaaaaatcatcttttttttttttttttttttctgagatggagtctggctctgttgctcaggctggagtgcggtggcgtgatctgtgctcaacctccacctcccaggttcaagtgaatctcctgcctcagcctcccaagtagctgggattacaggcacccgccaccacgcctggctaatttttgtatttttagtagaggcgggttttcgccatgttggccaggctggtcttgaactcttgacctcaggtgatccacccacctcggcctcccaaagatctgggattacaggtgtgagccagcgtgcccagcctcaaaagttcatctgtgagccacatttagaaggtagataaaaaattttgactaaacacagcttagaatctataaattgttaaacttcagttctgttgagaatctttgagatccatcattctctaaatgttttctaccagagaaacaggacaatggcttgcaaatattggttatcagtatttactctatagctcaagcaaacaaacttatttgcacttaaaatacaaatattacctaccttgatttgagtgttgttgttctctcttaaatttcatttcccaaaaatgatggcaaaagtactgtttgttttaacttgacccatcttttgcaacccagcttaatcatttcacagattcccttttgaaataaatgggctagatgatcagacaatagtggcaaatgtttgtcatttcaatgacatatgcaagagatacctttagtgaaatttgtatatcaaatttccctttgtgctccttttttccctagtgaattcagcattaaagtgtttataaatatttttggtttattatttattaattcaaagcaaagaatgccaatgtgaccataaatatctcatttatatatcataaatatctcattatagaaatatgtttagagacatctgttttattattttaaagaagagtacatattatatattaaggtaaaaatagtactaaaaaagagaaggtagtaaataaaaaaattctaatgaacataaaacatcataaaaattaacttaattaaaaatcagccaggcacagtggctcatgcctgtaatcccagtactttgggaggccaaggtgggtggatcacctgaggtcaggagttcgagaccagcctggccaacacggtgaaaccctacctctactaaaaatacaaaaaattagctggatgtgatgggacatgcctgtagtcccagctacttgggaggctgaggcaggagaatgccttgaacctgggaggcagaggttgccctgagccaagatagtgccactagactccagcctgggcgacagaaagagactctgtctccaaaaaaaaaaaaaaaagtataaagataaaattttagtatattgtgaattaattactagactcaattgttataggaagctttgtttttctggagctcttatattaaaattattaaaatcctcactgaaaagtataacatgtgttttctttaaaagaaagatattctatatctgaataaagacaaactttgaacgagtggtatatgccttaataaatattggagaatatctattctccaatacttagagaatgacgacagcatcctccacatggcgttttcattgccagtcacaactttcctccacctagtgttctctagactgagacagaccaaggaacaattccctagacctcctttgcagagctaggttatgctcagaccatttgcagcaccaaggacactgccattcaggtcaccaaaaaaagatcatcttgtgagccctgtctaaaccaggccccttctaaaatcatttcagtgtgaaactgagtccaggtgggtcttcatcagtattagagtggacctgccagatgtatgcttgcagatgatgcaacatcaaagcaaatgccctgggcaagagaagaaaggccaaagcaatgggcttactggtttctagctttgagccactcttcatagtgccaaattcccattttagggtccctttatttgtattaataaaaataaattccctgggtgtaagttcctgtaaagtactccctatctgtattcctgggcatgaaagggtgttttagttccaattctagttttcccacttagaggttcacctttctgacattaataattaaccagattatctgaattcttcaagttcagtttatttaaaaggagactatcctcaaaagtgttatatccgtggtttcctgctacctccaaccatgggccttttgggaatactttgttttttaatcttcctggggaaaacctggggacaggagcaaacgtaagtaaaacaagctatcttcatttttcaatgcatcttagttatgacgaatgaagagttaaatttaacagattctgttaacttgaaaataacttgctacttctgagaacagcaagtgaatagtatcatctctgaatgagaaagtcatggaagcattttgatttgatgagtagctaacaacatgacagcttttatggaattgttatttattaaaatttacgttaagtgattattcaaattcctcttcaaaaaagcacaaaagaaactccagataataaactttcttgacattttttgcttattttaacttggtgtttagttttctttgtgtttttcatagactatacacagttaaaccagggttatttaggagtcaatctaatactgtgatctgaaaatctgttgttgtagtgacttctaccatacatttatgagctgacttttcacaatttttagtgtcattattgagataatatgcattttaagtaatgttttaaaaataatgatcttatatctagaaatattttaatactgaaaagtaactgcagatcatctaatcaaacctattcattttatatatgagaataagtccagataagggaagtgactgaccaaaggtcacagttctagagagagagaaacaggatttttattaaggtctcctgtttatcacttcaatttttttcattttgtcataaatgtatacatgtaaatatattatatataaacacatacttaggatgtcattcttttttagcaccctcgtatttctgatctgtgatctttttacatggccacaagcactcaactaccttagcatcctgcctttattctttttgaaagacgagcagctaaggcttagagagactgaaattaagtgacttcaaattggtggcagaactagggctaggacctgggttccatgattcttgtccagacttattctgaattctttctactagcccttaataaaaattatttcctcactttctctgaagaagtcagagtgtaccatttacaacatggcaatcaatcactttaattaattggttgattgattgattcaacaaatatttattgagcatctaaacatatgataggcactgcagatatagcaaacggtaagacaattcctagagcttgcattctagtgggagaagcaagtaatgaatgaatgaaccaatgaaagaatgtgtaaggtcaggtaggtagtattatgaagaaaaaaataaagcagagtatgagaattaaaaaataattatggggcactattttaaaatgtggccaggaaaggttctcttaaggaggctgcactcaaacagagcctgaaggaagtgaggaagcaaaccatgcaaatatttgaggaaagagcattccagatagaggccaaaataacattcttgcctctctgcaagctcctgctcctctgcagcaattgaaagacaaagaaaactatgaaattatttatatggatagattatgctactgataagtcaataagaaccaaaatctttataaagggtggttccagaatattcttttaggggcttctaaacactatgtatttttgcttgacggctaccatcacatagaccagcagtccccaaccattttggcactagggacaggttttgtggaagacaatgtttccatggaccaggattggggtggggatggtttcagaatgaaactgttccacttcagatcatcaggcattatattctcataaggagcttgcaatctagatccctcgcatgtgcggttcacaatagagttcatgctcctaggagaatctaatgccaccactgatctgacaggaggcagagctcaggtggtaatgctcctttgccccactgctcacctcctgctgtgtagcctggttcctaacaagccatggactggtatttgtccatggcccaggggttggggacccctaccatacacaattcaaaattaaagcaatactaaacttaaaaacataaagcttctataaatactgaaattaaagtagctttatatccctaaatttttctaattacaaaactctttataaattccatttagtacaacagttgaaaatgttaattctggaaacgggctgctcaggttcgatttctaattctataatttgtgaattatagaattcataaatcctatatgtgtggccttgggaaaattaattagcagtttcttcaccttaaaattggagtaatagttcctcacagagtggttgtaatgacacaatgagtttatacaaataaagtattcagaatagtgccttatcacagggcaaaatttcagtaaatatatcttattatcataaaaactgaactcctctcattttctgccatctcctactaggcatgtggggaaggggaattcagatgatggttggaaatctggaaattctttcctctcttttgtaatttgcctgtaagtggatccaaattaattgtaatgcaggataactgtcagatgctttaaaggatttaattgctgataaattaattccattgttactaaaaacttcctgcttttaccagactcttaaaatgtcacaatgttggcagaagcctggaataattctgccaggtggggtggtgggtgccctccaagcttgttgggccacttgaattagtcgatcactatgcccttttgggttaaaacacttttttttcctatgtaatagcgtatgtagttctcgctattcaaattagatactcttgtgatatacacttagtaatccagtttatatttgtaagtttttaaataaaataatttttaaaaatcactcaaaagcatgtatagactcacttccattattgtataaatcacatgaaaagtaaaaataagagaacagtctatggttgtgaataacatataaacttgacctcaactgtacacataacaactgagaactcatataaaggcctcccccatttacttacaaaccgtataaccttagccaagttagccttttaccacttgcaaagtacgaataatgatagtaccagcatcattggcttattgtaaaaattaacaagagctgtatgtaaagtgctgatacagcctctgatatgtggcaagtacttaattaccattattgtttgaagatgatgatttgctcttaaaggaccatcatcactatttccatttccaaatgcacttcccagctcactcatattctatcctttccgttatccgtttactagtataacatagtatttctaagatcactccctttaaactaatttagtgagattaataaatcttaacatgtattgagtactaagagtgctagaccttttcatatatatgttcttatttaatctttaccagagcctggtaaagaaggcgattattttcacagtaagttattttagcctcagaaagacttagtgactttaccacggtcactcagccagcagatgccacagctggaattcaatcactgcttttttcttcattgagcaaaaaatattgaccccatttacatgttctggtcaacatgattaaaagtcttccttggctgcagtagctaagagaaagtgctgctatcaccttaactttctaaatgtaaactaaacctccagcttctcaggtcttaattccaaactacttggaaattatgctgaagttatggtgggtcacaagtacattttgacttattagtatcatttttaagtgaggaaatatatgtattttaaatggaatctctcaagtgagagaaaaaaatcaaaagtaaaagaaaatatgtcatataaagacattgagccttatcttttcctttttcaacagatatgtcatttcagcaccaaaaatattccgtgttggagcatctgaaaatattgtgattcaagtttatggatacactgaagcatttgatgcaacaatctctattaaaagttatcctgataaaaaatttagttactcctcaggccatgttcatttatcctcagagaataaattccaaaactctgcaatcttaacagtatgtatttatcctttattttctttatatatatatacagaatattctcaataaaaatgtgtacaaagtttgaatattgataacatatagatttaatcatttacatccactgtgggattattagatgatgtcagtcactaatgataatgtaagtgaaaattctaacagaaacagaaaattctaaacagaagttatgtctgctttttgagcaatgcatgtccattttcacattcttattggaaaataaaattgtactaatgtgaaaatatttggagataattgaaaatacttgtttgctatgaaaattactattagaaagaaattgagaaatatgagtatgattttatctgtagtacattgcccacaaaaatccatggatattattgaacattcgattgaactctaatattctacaattatagtttcaaaaactaaaaaatttgtgattactaaagtgaatagctctcattgacaacctgcagttaatagaaagaacaaccttgatcaaataactctcctattaagatattgtagtatagattgcttagagacatacttgcaacttatttttactaaaaattctgatacttaataaagaaaatcaaagaatatcagacgtaaaatgggctctgcagttcatttagaccagagtttctctaaagtgagttctttataaagaaattaaaaatggaaatttaataaagagcttaaaattcgaattatattgattttgtaacattttgaatttattttataaatttgttttgagtttttaattatacaaaaatcataagcataagattgtaccgttttaagtttgtatatatttaagtatctttgtatcaaaaattattttgattatgttataaataataaaaagccaatgctgagggtcctccatgacttttgtcctttaaaaggactatgcatattactcaagtttaagaaacactaatctaaaaaatttctagtcaaacataggaattctttaatgttcttagcagtaaatgctcatatctcttaacaaattcttccacttattaggtttgttaacaattaaaattcatttattaaaagtgttacttttatggcatatgcctgtaatcccagctactcgggagactgaggcaggagaatcgcttgaacccagaaggcggaggttgcagtgagccaagatcgcgccactgcactccagcctaggcaacagagtgagactccatctcaaaaaaaaaaaaaaaaaaagaaagtgttactttcaaaagacactcttttatctgatacaatgtatttgtggctcttagttttaaatttaaatcaaacaaatttaactgaaattaaaaatgcagtatctcagttgcactagcaacatttcaagtgttcaaatagccccaggtagctattggctaccatagtaaacagcatagatatagaatatttctgtcatcacagaaagttctctgaacagtgctgacctatatgattaactgaaaaactagattaaggtggggcaatcatagtcaatacatattttaaccatttaattttatcatagcaatatcatgtatctttatttgccaattgtttaatgttgtaccaaggtcttttaagtgtgggtctatggattgcagtgtcaaatctcatttattacgtgctacaaatttaatggatcatctttgttctatagtttcttttttgttgttgttgttgttgttgttgagacagggtctcgctctgtcacccaggctggaagacagtggtgtaatatcggctcactgcagccttcgcctcccaggttcaagcaattctcccacctcagcctctcaagtagctgggattaacaggcgtgcaccaccacacctggctaatttttgcatttttaataaagaaggtgtttcaccatgttggccaggatggtctcgaactcctgacctcaggtgatccgcccgcctcggcctcccaaagtgctgggattacaggcataggttactgtgcccagcctgttctatagtttctgtttcttaaaaggggagaaagaacttaaaggcacttacaaagcaatctgggtgcagaatatttatagatttttcttgttttccaaacttttacaataatttcgcatagctaattctacagcagttggtttttgtaattaactttcatcccgtgtttccaaagcctaatttttcatagatacacttttcttttttatcacttttcatgcattaaatttacatatcattgaatgtaaaggggataattacaaacataaattggaaaatgagcatctcaactcaggggagcagaaatatgtgaggtaatactaagggtccagagatgctggacacatgggcacggtgactgatgcctgtaatcccagcactttgggaagccgaggcaggtggatcacttgaggccaggagttcaggaccagcctggccaacatggtgaaacccgtctctattaaaaatacaaaaaaaaaaaaaaaaaaaagttagctggacatggtggcgcacacctgtaatctcagctacttgggaggctgaggcaggagaatcacttgaatctgggaggcagaggttgtcgtgagctgagatggcaccactgctttccagcctgggcaacagagtaagtgagactccgtctcaaaaaaaaaaagctaagggtccagagaatcggtcaggaggtttcacaaagggaatggggagcatggggtaacccagcaaactaattgagtgaaggtttcttaaaggcttttatagtagtagtttcactaaatgtatataaaatttaggaaacatggaaccaaagagactttcaattgcttaaagctaaatttttattatgtaattagacactgaagggcttcgggcaaaatttatttactcttcttgctcctgtatcactttttaaatgattacatatacatactgattgttctaatgagattacagttgtatttagaaagttaatggtttaataactttagaattattatactttttccatcaacaatttcttttccagatacaaccaaaacaattgcctggaggacaaaacccagtttcttatgtgtatttggaagttgtatcaaagcatttttcaaaatcaaaaagaatgccaataacctatgacaatggatttctcttcattcatacagacaaacctgtttatactccagaccagtcaggtagattataagatatttcttcctctgggtgggggatttgacagtattgacactatattgtagagctttttaatacctgaaattgagtggagatcggtttctttttaatttgctcaaaagactctcaattcccaaagacatctatacttacaaatatttaatagcattttaattttaaatttatggagaaagtgctaaataaattacaaaaccaacaattacattacctgccccatcacttatttccaagtagaaaatgctttttcaaaattgtaaaatgttgaatgttatgtaaaaatgtaaaatgttatgttgttacaaaaaccaactgctgtagaattagctatgcgaaattattgtaaaagtttggaaaacaagaaaaatttgttttccaatatgttacaatttattaaacaaatataagacttaattgcaaatataatttggcaatgggaaaatataatttattgtagaaccatgttcattaaaaattccactgggtctggagctaatccccaaactggaaaaataaatctaacctaggggatcagatatttcagcagaggataaactagatcacagaaatatatggatattaattttcaaaggggatctacctaagaaagttactgttagaacattgtttaacattcatggttcttctgttagaggcaaaagggcagaatagccaatcaaaatcatgtgaataggaatgactggacctattcccaatgaaccaaaagtaaaacacagaaaagtatgaatgcttcccaatatattcatgtgtttgcaaagccttccattcccccgtttctccctctcgtcatcaatgacttttaagtacatcagaaatagaatattagagatgtatatatgggaaggagaggctatggaatttcattaatgatattcttggaggaggacagagaaaggaagtggggaccatctgactttgttgaaaaactttgtacacagtagatatttgctaaatgttggttgatgttgatgatgagaatagtggggtggaactggactgaaccattaccgtttcagcaggagcatttgttatgatcagctcagaaaggaaaagcctaaagggggtggagatattgctatctacttggccagaataaaatctccagaataatagcatcttttttctccaagagacaactttttatacaaggaaatagtctctgcccacaaaggttgggtccttttagacataatcatacctagaaacaagcatatcatctctcttgtcctcctcctacattgaaggattcttttttgcttggatcctgttgacacgtcccacaactaagggtcatcagtctttgtccagtaagcagtgtaatatccagaggctgggaccttcctcggttatcaaggctttccttaagaatggcttcatgtctcatgcctgtaatcccagcattttgggagtccgaggtggggggatcacaaggtcaggagtttaagaccagcctggccaagatggtgaaaccctgtctctactaaaactacaaaaattagccaggcactgtggcaggcgcctgtaatcccagctactctggaggctgaggcaggagaatcgcttgaacccaggtggcagaggttgcagtgagctgagatcaagccactgcactccagcctgggcgaaagagtgagacttcgtctcagcaaaaaaaaaaaaaaaaaaaaaaggcttcatgtattatttctctatcaggactttactgtaagatgatgtaatacctggccaggcacagtggctcatgcctgtaatcccagtactttgggaagcccaggcgggtggatcacttgaggtcaggagtttgagaccagcctgaccaacatggtgaaaccctgtttccactaaaaatacaaaaattagctggatgtggtggtgcacgcctgtaattccagctactcgggaggctgaggcatgagaattgcttgaacatggcaggcagaggttgcagtgagatcgcgccactgcactccagcctgggcaacagagcaagactgtctcaaaaaaaaaaaaaaaagagaaaaaaaaagaagacgatgtaatatcctcatccattctaatcgtagagtaaaatgagagaagtttgctataaatattggcccatttctgcaagatagtgaagtaaattggttaaaagcagactctggagtcagattgtctgggttcaagtcacagcttgagctgctgtgagctgtatgaccctgtgtgaatcactcagcctctcagactcccactttcctcacttgtaaaatagagataataatacaacttacctcatagagttgtgagaattaactgaattagtatgtgtaaagcacttaagatagagccaagcacacagtaagcactaggaaagtgttttccattattatctatggaatagcacaaagccatatttacagaaaagaaaaataattacttaaaagtattttttaatcgtcttgttatttatagctataatgtcccgatcaacacatttaatctgaagattcttgagggatttttagtgtaagcatctttaggataaaggcttcaaggctttactggataaatgactttagaatattgcccaaaatccttcaatttcataaagcctgtgtcctgccttcttccccagcttcatcacctactacggccccaccctctatagacacaagccaaactgacctcctttcagatgctttctcacaccgtttccccatccatattaaggcctgtgtaacatgctgttgcctctacatggtacattcttgcccctcctttgccttgttaactcctacttcaccctcacgtgtcatcacagatccttctaaaggcagctttcgctgatttccctgacctcttcaaatctccctcttctacatgctcataaggccaggcaaatatcctttgtggcacttcacaccctagaattttacatataattgtgtttgcctctttcctccacaccattaaaagatgtccatgtctgatttgtttcactactgtatctcagtacgtagcataatgactggcacattagaaggcattcaaggccgggcgcggtggctcatgcctgtaatcccagcactttgggaggccgaggcaggcggatcacaaggtcaggagatcgagaccatcctggctcacacgggatgtgagctaatacaaaaaattagccaggcatggtggtgggtgcctgtagtcccagctactcgggaggctgaggcaggagaatggcatgaacccaggaggtggagtttgcagtgaaccgagattgcgccactgcactccagcctggcattcaaactttttttaaattaataaaccacatcttttatttttatttttatttagtgagtgtctcgctctgtcacccaggctggagggcagtggtgcaatcttgtctcattgcaatctccgcctcccaggttcaagcgattctccagcctcagcatcccaagtaagtgggattcaggcgcgtgccaccatgcctggctaattttgtatatttagtagagatgggatttcaccatgttggccaggctgggcttgaactcttggcctcaagtgatccacttacctcggcctcccaaagtgctgggattccaggtgtaagccactgcacccggccaataagccatatcttttatacaaaaaattgtatagatcttgcctccaatgtaccatcgctgtgaagacaagattaattttctaacctaaacacaaactgggttaaaaactagccaaatagtagtaacagaaataataataaaataaacatttattgattacttagtatgtgtcaggctatgcaaaagctttgcattatttaattctcacaaggacactatgagcacacactagtattcctgttttacaagtgagttaactaaggtttagagggattaagtatcctgctcagggtcatactaaaaagtggcagaggcaggagtgaaaccaaagtgtttaattccagggcctgtgctcttaacttttgtgactttctgacttgggcagcttcccagtgatactcaggcctagactgggtgtgtcccaaaaagcaggagataggatttgtggtgggagacaggtgtgaggggaaggagagagcagagaggtggggcatttactgagcacattgtttttatcttgtgaacttagtaaaaccagtgttaatgtggcttcttatatagagctttgcataatactgatcttttaaaagatactgggtttatggtgccagttctattttattttgtgggtaaagttttggccatgaaatgatttgagcatcagcaacaccctagagaaattaagtttgtcattggaaagaattagatcactgtgtctcaaacagctgtgagttaaaagcgaaacagatgcttttgtttaaaaagtagggatctattaactgacagaaagaaatggaagagaactaacagttattgagcccctgccattgctcttttaacaatgagcgaacaagtgtaggatctggctcaataaatagaacaaaaccatataatccaaagctgccttttgcaataagagctaatttggagccttgtgggttttcataagtggaagagtgtatgtctgaatcaagctcccaattttccattaagatcttcaactctgagacaatatattatgctcctctataaccatatggtgatgtcactattttgctactatgaaattcttaataaataatggcctaattacagatgtaattaatttcatactgtgaatgttttctcaaaacattgtaggtaaatgtattttaagaaagtgaactgtactttaaatccactaagagaaatagttctttaaaggaatccctgaggattctcttaaataagagaatctttagtaatgtatatgtctgcaaactaatttttgtttttttcaaatatggacttttgtgtattgagttttttaaagtaagattcatctcttttaaggatggttgttttaaaaaatcaaaatatccttgtttttagcagagtttttgattatatcaattattcttacagtaaaagttagagtttattcgttgaatgacgacttgaagccagccaaaagagaaactgtcttaactttcatagtaagtattttatttatttaaataatactttaattcatttttcaggacaagaatctcatttcataaccctgtcctcacactacactaaatcattcaatgttccataaatggttattgaacatctgcaatgagtatagaatcatagatctatacctctgtaatacacgtatgtgaaaggctcatacaaattaaatttcaataacttgaaggattttgagctaaaccatttaagtacaatcttgctgatttattttttaaagaacacccactggccgggcgtggtggctcacacctgtaatcccagcactttgggaggccgaggcaggtggatcacgaggtcaggagatcgagactaacctggccaacatagtgaaacctcatctctactaaaatacaaaaaattagctggtgcagtggcaggcgcctgtagtcccagctactcgggaggctgaggtgggggaattgcttgaacccgggaggcagagcttgcagtgagccgagattgtgccactgcactccagcctggtgacagtgtgagactccatctcaaaaaagaaaatctaaaaaaaaaaaaaaaaccaaaaaacaaaaaaccacccaccaatcactaagaattgactgaatacccatttaactataagatatacataatggatatatctattatgtatttattatgtgcaaaagaactatatttaatggtacttactcttaaggaacttagagtttaataactattatactaacacaaaaaatcttgctaaatcatgagttaaagattggcagtaattcaccatttatcagacatgacatatgtaacagacgctgtgcattgaactcccatgatctcatctaatttgtgtcacataccccaaacacttttgtgaggaaataaaataatacaactagttataactgctcagttattaacagtaggtatactactaatggtccttcaatataggctgcagcacttttgctgctactcaaaaaaaaaaccaaaaaataaaaataaaaacttatcagaatgcaggtgcaaaagcatagatactacagaacaaaaccatactaaaatataaaacaaataaattatacagtaggacaaataccatgagctcagagggaagaccaatgagaggtgagagtgggggtgatacacatccttctctggaacggagtagttaaagggaaccctgagttgggtgttaatgaaaggaattgacataaattatttaaaaatagtgaagaccatccatgtaagaaacagtgtgagttaagctatgggggcaagaagaaataaaaaaaaatgaagctagtaaataaactgattgactgatagaatgtctgaaagagaactaaggagagcaatgtcagctcaaatgccctttttctatcagggaatgataggggtgtggatgtatgctctttatccaacgtcttcatggttgaacttaagtgatgcagaatcatggcataacctgcagaaaaatgaatgtcagtcatctcttgtaagagctgccccaggaagaaaagattgagaaagctgatcccagagccttaaggagttaatatagaggaagtatcctcaaaatagtacctgagaaagatgatcttagaagaattgtgcacaatggatagtgcgatgaagaagagccataaaaatactcagctacaagtataattcttgttcaaaataggcttctgtaagttttacaattaattacgaaataaaacctctaaaatccaactgtgaatttcagtcatggtaaatggtgctaagattttttgctttgggttttttgttggtttgtttgtttttgtttgtatgtttttgacagggtcttgctctgtcaccaaggctggagtgcagtggtgtgatcatagctcactgtagcctcaaactcctgggctcaagccatcctcccacctcagcttcctgagtagctaggactacaggcacccatcaccacaccctgctaactctttaaacatttttttgtaggctggtcagtgactcatgcctgtaattgtaatcccagcactttgggaggctaaaatgggaggattgcttgagctcaggagattgagaccagcctgggcaacctggagagacctcatctctactaaaaattaaaaaaaaaaaaatagctgggcatggtggcatgtgcctgtggtcctggctacttgggaggctgaggtgggaggattgcttgagcctgtgagttcaaagctgcagtgagctatgatcacaccactgcactccagcctggatcacagagcaagatccctgtctcaaaaaagttttttgtacagataggggcctcacttatgttgcctgctatgtttatgtgttgagttaaggctgaattggacccccatgtgtagtccaaagaaagtcaaaacctcatttcaattatttgctaacccaaacacaaaattaagtttatggggaagctttacgagaactgtcaagtttgtttaaccatttgagagttttaccaagctgaaaaatgcccatactatacatttataaaacatctggctaaaaaatataattcatagaatatttgcttttgaaagctttaaatgaacatatataaaaattagattctggtgtttataaagccaacaaaattcacatttagtttatcaaaaacatcattgtttgtttgtttgtttgtttgttgttgtttttacgtgttgttacacttcatcttttagatgtacaacgagagttgagtctcttcaagataatgaggagagatggggaagattggaaaaagaagaagggaaatggctgaaatgaaccatatttcccttctataacatttaacagatcttcatccttcatatttaatttgaagacatgggtatttgtcttcaaatatttgatctataaatattttgtacatcaaagattgtataatcagttaaattttttaaaatgtagtttctggcctttgagggtgtttgtttttatttacaggatcctgaaggatcagaagttgacatggtagaagaaattgatcatattggaattatctcttttcctgacttcaagattccgtctaatcctaggtatgtaaaaatagttgttaataaaaccacatacggaacttttggtgaagaagcaggtaaccagttaaagggggccacctaatgttaacaaagggtgtcacataggtggatatgcaaaatcttttgaagttctagcaagaacttaagctctgttcttggcaaaaaatttccaatacttctcagccattctttttacatatagaaataaaaattgggaagaggaaaaaatagtacccaggtttttttgtcataaaaaacatcaactcctacccctcaaagtatctaatagattgtcagtttaaaaggctggtgtggtcttgttccttgctctggttgctataaaatagggtcgtctgtagtttttgcctggataaactaccagaaggcaaagtccaagagggaaggagttctcagagctatatctattatttggcctcacatgaaaggatttaagaaaaagtttaaacagggttcaatgtaataaatattcaatgaacacattcttggtatgtgcaaatcacaataagagggagagatacaaaataaataatccacagtccctgctttcaagaaatttgtaataggccaggtgcggtggctcatgcctgtaatcccaacgctttgggaggccaatgtcggtggatcacctgaggtcaggaatttgagaccagcctggccaacatggtgaaaccccgtctctatcaaaaatacaaaaaaagccaggcgtggtggcaggcacctgtaatcccagctactcgggaggctgaggcaggagaatcgcttgaatctgggaggtgagggttaaagtgagccaagatcatgccactgcactccatgttgggcaacaggagcaaaactccaactaagatagaaagaagaaggaaggaaggaaggaaggaaagaaagagggaggggagggaggaaggaaggagggaagaaaagaaagaaagaaatttataatagagcagaatgaataagacaagtgcatctatacatggctgcactagaattttagaacaacatctttttatggtcatttttcctctgccctcagttaattcgctgactgaaatttttctaaaaatttaaattgaatttggaggttttaaataacttggataatttaaaaattgtgtacattcttgaaaatactacatgttccatagtgcagaattaaacctattattttattactgctatcatttctccaacttcatcttgtgccacttgccccatggcaaattatgtgctagccatggtctggcatgtccagtgctttcctacctccgtgcctttgcacatgtcatcccctctacttggaatgggcttcacctggctcttcataagcctggcttcttttcattgttatgacccagagtagaagctactacaaaaaggctattcctggatgcatgatgtaatgtaatctcttgctttgttttactccctatctcagcatcttgtttattttcttcatgacacttatctcaatgtcataatcatttctttacttgcttgctggcatttttattccccactagcttataaactctttaaggtgggccttgtctgtattgcacaccagcagttcaacataataggcccttgataaatattcattgaattgatgatgatggtaataatcattattataatactattaacatttgtggacttcatagccaatctgcaaggtaggctcagtatatatatgtgtgtgtgtgtgtatatatatgtgtgtatacacacatatatacatacatacacacacacacacacacacacacacccatagtttttttgaaacagagtctcactctgtcacccaggctggagtgcagtggtgtgatctcagctcaccacagccttgatctcgcgggctcaagcaattctcccacctcagcctcctaagtagctgggactacaagcatgcaccaccacaccagactaatttttgtatttttttgtagagatggggtttcatcatgttgcctgggctggtctcgaactcctgggctcaagcgatcctcctgccttggcctcccaaagtgctgggattataggcatgagccactatacctggcctagtatatgtttttgaataaaggttattaattaaagccaggtcattttcatcttttaaaaaccatgctttgtttccaaccataaattcatttttgtgaggtgtaaattttatcatctgtataagcttgtcttgtattatatgtttctagagacgttattatggattttgtactgtattttggattttagtattatgttagcataattaggctccaagattaaataaattctcagaaataataagttcttcaaatcaaaactaatgactgtattaagataaaataccaggaaaaataatacaatatatgaaaaactggttactaaacaatatctattatctattatcctaaaaatgctcaattatttattgcttaaaaatcagtcacaaataaaatatattacttaggttttatatatctttgtgtattcttccctctttaaaacaggaatttattagatctcctctgagtgaattaaaaacatttgatgacatctataattttaaatattttgcttccacagatatggtatgtggacgatcaaggctaaatataaagaggacttttcaacaactggaaccgcatattttgaagttaaagaatatggtaatttctgacaagtaaaagtttttctttgctttttgttttttgtttttgagatggagtctcgctctgttgcccaggctggagtgcagtggcacgatctcagctcactgcaacctccgcctcctgggttcaagcaattctctgcctcagcatcccaagtagctgggattacaggcaccaaccaccatagccggctaatttttgtatttttagtagagacagggtgtcaccatcttggccaggctggtcttgaactcctgacctcgtgatccacccacctcggcctcccaaagtgctgggattacaggcatgagccaccgcacccagccaattaaaagtttttctataaaaagttagtgttttgcaataattctttttaataatgtagttaatgcaaaattattcccagatgaagatgaggcagcctgtacgtatggctagaagtactggctagaggttggagggaggagtagtagttgaatgcctagattctagttccagcatgcctttaaccctgatcaagcagaacaacctctgtgtacttgtgttccccatcagagagctggaagagatctagtcccctgaagagcttgactagtaaagatcctttctagaattgtgattattattaggagaaacttaaatagtcatagatgaggtgagtggaacccagtaaaaccaataaatgatttacttgagtcttaaaaccaattagtgacaaaacaaaaacaacaattaggtttccttacccctagtccaacactattttctggctcctaaattcctaatatattcatttgaatatttaagatattcttattttgaaataaattatattaatagactaacaagtaacgtaaatagtttaatctttcatttcttatagtagtttactagcatttctgtgtgctctgctatcaaatttaactttcttctgcttgaagccattttttattccctcaagagattcataatgacatggagaagaataaatactgacacaaattttaacaattaacaagaaaaacagtgacaaaacaaattcgttatgatgaaactcaaccttaaagattacaggctctctgtaacatataatcctaacccatctcccttcttctaagtgtttttgtattttctctgtatatctagaatccacccacctcttactgtctgtagccactctcatcaattcatcagcactctccctcctggactacagcaaaagcctcctaactgatgtctctgcttccaagctatcttgcttggtattagccaaaataatcttgaaatgcaaatttcattatgttatgtcacaattgaaaagtttttttgttttcaaggaacctatactctaatggaagaaataacctattgcctataataaatgctgtaatcaaaatataggcaaagtgcttctggattgacctctgattgttggagtgggaaggttctgcagagattgttgttggtgatgacgatgatgacgatgatgattatgataataaagtggatgttgtgttgatgttacagcttgtccaactataccagaaagtctagctgtttgctaggctttaaagtctgctctaatcaaaatggtaatattacctgtttgtttttattttcagtcttgccacatttttctgtctcaatcgagccagaatataatttcattggttacaagaactttaagaattttgaaattactataaaagcaaggtaagaacatttgtttttttgttgtttttatttttatttttcaaattaactcaactctggttgatgatagttgcccaaagtgagatctgtgatttattatcaaagtgtgccatgatttggggactaattgataaaggagacatcatgctggtagttcatttgcttattctgattttagctgattctgagtttagctttgttggctattcaacagcaaaaacaaagaaaagtgccaaagatctaagtttggtgtatgtgtttagaaactagggtaacttagaatattgggactgagatatgaaggattcacttcctatctttttttaatgatttaatcagccagctaatgctgcgtaactacttcaaaacccagtgccttgaaactcccacatttatgagtcagctgggaagttctgctgatctgggccaggctggtgtcagctgggctcacccatgtagctgcagccagttggtaggtcagctgggggctggctggtctaggaaggtcctgtccatatgtctggcagttggatggggctaaaagtgtgtgtgcctctcatcatccagcaggctagcttgtagcagcagcaacactcaaagaggcaatggaagcatgaaagtccccctgagcctaggcttggtgctggcacaccatcacttccacacattcttttgtccaaagcaagttacaaggtcagcccagaggaaatagacccctcccacactttgtggacacaaggaactgtgcaaaggtcgttgatacatggggaggtagagaattgggggttagttttgcaagcaacctatcacaaaagcttaggtcattttaaaattacaatataatgtagttataccaatctatataactatatgtgtggggtactcttctaagtactcagcatatattagcacatttaatctttacaacaactctatgcgttgatattgttactattcccatgttacaagtggaaaaactgaggtacataaatgtttaataatttgctcaaaatcatacagctagtaagttgcacagcctgtctgcttccagagtccatgcctcaaacattaccctatgctagctctcttattagcattgatttgaaaaatagagtgtttattacttggtctaaaccagcataaaaggctcacttgagagaaaattatagaaactgacctcctataatggggactatttattcaaaccataaataaggaaagttagctcttgctatagtattatttgctgggtagaaagtttactttattcttactttaattctaaactaaaaagggaaacatgcagtttaaattttttatgaatagatctcatataacagtgagattggacacgtaccttttaaaaacataaaagaaaacagagagtagctgatatcaaaattatcaaggaagtaatttggcatataggagagggccctgattctaagtcttgtgatctccccaagatcaatttttttcatctagcacatagctttattaatgtggctttatgaggatgaaatgtaatagtgcatgaaggacactttatttattattattatactttaagttctagggtacatgtacacaacgtgcaggtttgttacataggtatacatgtgccatgttggtttgctgcacctattaactcatcatacattaggtatttctcctaatgctatccctccccgtcccccaccccatgacaggccccagagtgtgatgttccccgcactgtgtccaagtgttttcattgttcagttcccacctgtgagtgagaacatgcggcatttggttttctgtccttgtggtagttcactcagaatgatggtttccagcttcatccatgtccctgcaaaggacatgaactcatccttttttatgcctgcatagtattccatggtgtatatgtgccacattttcttaatccagtctatcattgatgggcatttaggtgggttccaagtctttgctagtgtgaatagtgctgcagtaaacatacgtgtgcatgtgtctttatagcagcatgatttataatcctttgggtatatacgcagtaatggaatcactgggtcaaattgtatttctagttctagatccttaaggaattgccacactgtcttccacaatggttgaactagtttacactcccaccaacagtgtaaaagcgttcctatttctccacatcctctccagcatctgttgtttcctgactttttaatgattgccattctaactggtgtgagatggtatctcattgtggttttgatttgcatttctctgatgaccagtgatgatgagcattttttcatgtgtctgttagctgcataaatgtcttcttttgagaagtatctgttcatatcctttgcccactttttgatggggttgtttgtttttttcttgtaaatttgtttaagttctttgtagattctggatattagccctttgtcagatgggtagattgcaaaaattttctccattctgtgggctgcctgttcactctgatggtagtttcttttactgtgcagaagctctttagtttaattagatcccatttgtcaattttggcttttgttgccattgcttttggtgttttagtcatgaaatccttgcccatgcctatgtcctgaatggtattgcctaggttttcttctagcgtttttatggttttaggtctaccatgtaagtctttaatccatcttgaattaatttttgtataagatataaggaagggatccagtttcagctttctatatatggctagccagttttcccatcaccatttattaaattgggaatcctttccccatttcttgtttttgtcaggtttgtcaaagatcagatggttgtagatgtgtggtgttatttctgaggcctctgttctgttccattggtctatatatctgttttggtaccagtaccatgctgttttggttactgtagccttgtagtatagtttgaagtcaggtagcatgatgcctccagctttgttctttttgcttaggatgaaagacactttaaagtgcctcaagtgctttacgtaatttcaaattgttacattttatctgaatcctgttattgtaaccatgttagtaaaacagttttatgctattgtctgacagatatttttataataaagtagtcactgaggctgacgtttatatcacatttggaataagagaagacttaaaagatgatcaaaaagaaatgatgcaaacagcaatgcaaaacacaatggtaagatgttaagacacagtcactcacacctacagatgcatccagaggtgagagatgagaagttacttaaaaaccatctagtccaatcccccattttagataggttgaaacttaaaatcctccattggcttccctttgcctttagggtaaagttcattttccttggcatcagcctcccttaccagctgatctcatgccctttcctccctccctcccctttgtattaatcactccaatttctctaactgaccaaagtctctcttgcctccaggtctttacaatatcatttattctgcttgaaaatgctctttcccattgtcacctcctgccccttctttgcctggctaattccttctcattctttaagtttcaacattgacagcctttctctgggaagcctcccctgactcttctacactaggttagctgccccggctgtatagcctcttttcttaacacattcttcacatttcattgcagcggttctcaactaggggtgattttttattcccaaggagacatttggcaatgtccagagacatttgtggttgctactggtgtttactagggagaggccggggatgctgctaaattcctaccatgcacagggcaggcccctacaacaaagaattatcctgttcaaaatgtcaataatgctgaggggttgagaaacccttctttactggaatggcttgtttacttatctatcattttcaatattaaactatttgtgagtctgaatcatttcccttatatgcccagagacagcatagtttcttgcaaggaaatattcatgaagttatttgtccatagtttagggctaatgattgctggaactgagattagggcgctggttccctttcccttattcagcattctttacattataccacagatgatatgagttcatgtcatttttgctcactttttcgtattcatttggtttgcatcatcaatcatttatcctctacaatcttattaattcaagatgtaaattaaatgcatttttaaaatgcaaaattctatgctagacactgggatgtaaagctaaataagacaaaaatgcctgccctcaagtcgctcaattttataagaaaaaataaggcaaagtttattttactgttgtgttaataattaaataatataatatgacttaaccccacatccctttactcattgtgtgttttaaaattagtatttaaatttgtaagcagtgaagtacaaattaaatattataaaaaccaacaaactctcagttaagggcggaagtacgctgagaggcctataaagatgaatgatgacgcaggaattggaagaagtcgtgtttgtcagctattaccaaaaagacctgttggaccatgtgatgttcaagacccttctacttctataattctatacaaataattagaaaataacatcctgggcttaatcgtggactcggttatctactttacttttgacactaaaccattcttctgacatttaccttcaatttagaggttagtaaaagcagattttattaatttttccttataagttcttctccgaaataactcctcctttttctccttttaaaaagttgataaatggaattgctcaagtcacatttgattctgaaacagcagtcaaagaactgtcatactacagtttagaagatttaaacaacaagtacctttatattgctgtaacagtcatagagtctacaggtaagtgtgtgtgtgtgtgtgtgtgtgtgtgtgtatactttcttttgaaaaatatttagacagaatattaaagatataaataatttccaatttctataattatttcacattgtattttctttcatagttaaatataattataagaagtagacttttatatagttataaaaaggcagtagggctctgcttctttttagtcattggacaagccattgggtccctattacctccacttctactgcaaaaatgggaaaacagtacccaattcatgtccaaagtagttctgcaggctctctgccttgaccatttggcaccctagtgtcctcacgtggtgaaaatcaaaaccaaaataagattatgtgtgtaaaactatttgataaagtactatacaaataaaagttattactattatcattattatagtctacataatgatggagtcaaactgaatgtttcctaaagcctaaagttttcaacattattttaatgtttcttctttttattttttcctcaacttattttaaataagttcagtttaagcagtatctatgagatatttaaagtattgccaaacagacaggcttgcaaacattaatgatgtatgatgccagcactaaagtggcttacaaattagcatcaggtataagacaggtgaagaaagaactgttgcaagaaagaattccatatgagctctaagcaaaatattaatgcaataggattcagaggaaggtgagcttctagatgattgttgagcaagcaagaacagtgttgtgaagatgatcagtcttgaaaaataattagggttttgaggcagagttgctagacagggtgaaatagaaaccctccagagagagggcagatgaagaagagtagatggtgaagcttaatctttgaggacagcagatcatccagtttggctaaaatataggatatgtatagaatgtattagagatgtaatattggaaaggagtttgggaatattaaagcttcaaatgttattcttaatagtttaacttaatctttacctggaggataacattattgggctcttgccagttgtctggtcatgtccccttccctccccctcccccctcccgctcccctccctcccctccccctcccctccccctcccctccccctcccccctcccgctcccctcccctccccctcccctccccctcccccctcccactcccccctccccctcccccctcccctcccctccctccctcccctcccgagtcttgctctgttgcccagactggagtgcagtggtacagtctcagctcaatgcaacctccgcctcctgggttcaagcaattctcctgcctcagcctcccgagtagctgagattacaggcatataccaccatgcctggcactttttttgttttcgttttttagttttagtagggatgaggtttcaccatgttggcccggctggtctcgaactccctttcagggttctttctatatcaagctccctttttctgatttctctctatgtatctatgctctgtgaacaccaacccccatcccccacctccacttctgtaaggctcagtttctggcctgacccttccctctctcctcaatcccttgaatacagtcaactgaaactcaggccactgatgaaaagtggtgtcccttcctggcatccttcctcagtccagtcctgtagctctacttctccaagggcttctctacatacgagcttgtgcataaggccttggaaactcctgaggtctaaactcttctctgctgtgcccctctgaagcaagacctatgtcctacttactactggtaaatgaatgaatgcaagaatggagtgagtgaatgaataagtctgtggagacatatagagatgaaatggatatccatacagatcatgatactcctaaaaactgtcaacatgatgcttgccatttccttaggtggattttctgaagaggcagaaatacctggcatcaaatatgtcctctctccctacaaactgaatttggttgctactcctcttttcctgaagcctgggattccatatcccatcaaggtagattgggtgagggggtagacgttgctacatatgatcatcttgttctgttcatggaaaacatgtgggtggcaaacagggtaagaatgttgccatgaagtcacagagggagcttattctgtgaaacctgcccctcctggtggacacagacagggatgggaaattgaaggtcagctgcagccttggcaatgacttcaaaagtgagggcaagatagccttgtattaggcatccctaaataagcattaaattacatgtaataaataagcattaaattatatgtaaataaaatctcataagactctagttcaaaaatacaaacatttaaaattatttggtggcaaattcttaaaaagccttaagatggagatatcattccttgaaaatagactcctttctctcactatgtttttgcctccagtgtgtctcatttgt

(19) United States
(12) Patent Application Publication
(10) Pub. No.: US 2024/0417725 A1
(43) Pub. Date: Dec. 19, 2024

(54) OLIGONUCLEOTIDES FOR TARGETING COMPLEMENT C5

(71) Applicant: Vanqua Bio, Inc., Chicago, IL (US)

(72) Inventors: Kevin Hunt, Chicago, IL (US); Maria Nguyen, Chicago, IL (US); Daniel Ysselstein, Chicago, IL (US); Carole Deyts, Chicago, IL (US)

(73) Assignee: Vanqua Bio, Inc., Chicago, IL (US)

(21) Appl. No.: 18/700,445

(22) PCT Filed: Oct. 12, 2022
(86) PCT No.: PCT/US2022/077969

§ 371 Date: Apr. 11, 2024

Related U.S. Application Data
(60) Provisional application No. 63/254,808, filed on Oct. 12, 2021.

(51) Int. Cl. C12N 15/113

(52) U.S. Cl. CPC C12N 15/113; C12N 2310/11; C12N 2310/315; C12N 2310/321; C12N 2310/3341; C12N 2310/341

(57) ABSTRACT

In some embodiments, the present disclosure provides compositions of antisense oligonucleotides (ASOs) such as gapmer ASOs for inhibition of complement C5 expression. Further, in some embodiments, such ASOs are useful for treating diseases associated with complement C5 dysfunction. Where a sequence is complementary to an intron of a complement 05 pre-mRNA.

### RELATED APPLICATIONS

This application claims the benefit under 35 U.S.C. § 119 (c) of U.S. provisional application No. 63/254,808, filed Oct. 12, 2021, which is incorporated by reference herein in its entirety.

### REFERENCE TO AN ELECTRONIC SEQUENCE LISTING

The contents of the electronic sequence listing (V034570003WO00-SEQ-MSB.xml; Size: 297,669 bytes; and Date of Creation: Oct. 12, 2022) is herein incorporated by reference in its entirety.

### BACKGROUND

The complement system comprises more than 30 proteins that function to maintain homeostasis throughout the body of a subject and preventing infections (e.g., systemic infections). Misregulation of the complement system can be indicative of disease or a proximate cause of disease. Complement component 5 (also referred to as ‘complement C5’) is a principal actor in the late stage of the complement signaling system. Activated complement C5 assists in activation of pro-inflammatory immune cells, while causing destruction of other host cells by triggering pore formation. Certain diseases such as paroxysmal nocturnal hemoglobinuria (PNH) and atypical hemolytic uremic syndrome (aHUS) involve misregulation of complement C5. It has been shown that a monoclonal antibody that targets complement C5 (eculizumab) can be useful in treating PNH and aHUS. However, this treatment is expensive and does not function for all patients. Accordingly, new compositions for treating diseases associated with complement C5 are needed.

### SUMMARY

Aspects of the present disclosure provide compositions and methods for inhibition of complement C5 expression. The inventors of the present disclosure found that antisense oligonucleotides that are complementary to complement C5 pre-mRNA are effective for inhibition of complement C5 expression and treatment of diseases associated with complement C5 dysfunction such as paroxysmal nocturnal hemoglobinuria (PNH), multiple sclerosis (MS), neuromyelitis optica (NMO), neurotrauma, stroke, amyotrophic lateral sclerosis (ALS), Alzheimer's disease (AD), Parkinson's disease (PD), and Huntington's disease (HD). Thus, the present disclosure provides antisense oligonucleotides comprising a sequence that is complementary to an intron of a complement C5 pre-mRNA (e.g., intron 1, 2, 3, 4, 5, 6, 7, 8, 9, 12, 14, 15, 16, 17, 18, or 20 of a complement C5 pre-mRNA).

In some embodiments, the oligonucleotide comprises a length of at least 8, 10, 15, or 20 nucleotides. In some embodiments, the sequence that is complementary to the intron has a length of at least 8, 10, 15, or 20 nucleotides. In some embodiments, the oligonucleotide has a length of 8-100 nucleotides, optionally a length of 18-30 nucleotides. In some embodiments, the oligonucleotide binds to the intron and selectively inhibits translation of the complement C5 pre-mRNA. In some embodiments, the oligonucleotide binds to the intron and selectively inhibits downstream expression of complement C5. In some embodiments, the oligonucleotide is complementary to an intron of a complement C5 pre-mRNA, wherein the complement C5 pre-mRNA comprises the nucleic acid sequence of SEQ ID NO: 301. In some embodiments, the oligonucleotide is complementary to a portion of the nucleic acid sequence of SEQ ID NO: 301. In some embodiments, the oligonucleotide binds to a portion of the nucleic acid sequence of SEQ ID NO: 301. In some embodiments, the oligonucleotide binds to a complement of a portion of the nucleic acid sequence of SEQ ID NO: 301.

In some embodiments, the antisense oligonucleotide comprises a first sequence that is complementary to an intron of the complement C5 pre-mRNA and a second sequence is complementary to an exon of the complement C5 pre-mRNA.

In some embodiments, the entire length of the oligonucleotide is complementary to an intron of a complement C5 pre-mRNA.

In some embodiments, the oligonucleotide comprises at least one modified internucleotide linkage (e.g., a phosphorothioate linkage). In some embodiments, the majority of internucleotide linkages of the oligonucleotide are phosphorothioate linkages. In some embodiments, each of internucleotide linkages of the oligonucleotide is a phosphorothioate linkage.

In some embodiments, the oligonucleotide comprises at least one modified nucleotide. In some embodiments, the majority of nucleotides of the oligonucleotide are modified nucleotides. In some embodiments, each of the nucleotides of the oligonucleotide is a modified nucleotide.

A modified nucleotide may be a 2′-modified nucleotide (e.g., a 2′-O-methyl nucleotide, 2′-fluoro (2′-F) nucleotide, or 2′-O-methoxyethyl (2′-MOE) nucleotide).

In some embodiments, each of internucleotide linkages of the oligonucleotide is a phosphorothioate linkage, and wherein each of the nucleotides of the oligonucleotide is a 2′-O-methoxyethyl (2′-MOE) nucleotide.

In some embodiments, the antisense oligonucleotide comprises at least 10, at least 11, at least 12, at least 13, at least 14, at least 15, at least 16, at least 17, at least 18, at least 19, or at least 20 contiguous nucleotides of a nucleic acid sequence as set forth in any one of SEQ ID NOs: 1-150. In some embodiments, the antisense oligonucleotide comprises at least 10, at least 11, at least 12, at least 13, at least 14, at least 15, at least 16, at least 17, at least 18, at least 19, or at least 20 contiguous nucleotides of a nucleic acid sequence as set forth in any one of SEQ ID NOs: 1-150, wherein one or more of the thymine nucleobases of the nucleic acid sequence are replaced with uracil nucleobases (e.g., one thymine is replaced with a uracil, two thymines are replaced with a uracil, or all of the thymines are replaced with uracils).

The oligonucleotide may be a gapmer. In some embodiments, the gapmer comprises a central segment of 5-20 deoxyribonucleotides flanked by 2-15 ribonucleotides on either side of the central segment. In some embodiments, the gapmer comprises a central segment of 10 deoxyribonucleotides flanked by 5 ribonucleotides on either side of the central segment. In some embodiments, each of internucleotide linkages of the oligonucleotide is a phosphorothioate linkage, and wherein each of the ribonucleotides of the oligonucleotide is a 2′-O-methoxyethyl (2′-MOE) ribonucleotide. In some embodiments, the oligonucleotide is a gapmer comprising a central segment of 10 deoxyribonucleotides flanked by 5 ribonucleotides on either side of the central segment, wherein the oligonucleotide comprises at least 10, at least 11, at least 12, at least 13, at least 14, at least 15, at least 16, at least 17, at least 18, at least 19, or at least 20 contiguous nucleotides of a nucleic acid sequence that as set forth in any one of SEQ ID NOs: 1-150, wherein the thymines in the ribonucleotide segments are replaced with uracils.

The oligonucleotide may be single-stranded or double-stranded. The oligonucleotide may be complementary to at least 10, at least 11, at least 12, at least 13, at least 14, at least 15, at least 16, at least 17, at least 18, at least 19, or at least 20 contiguous nucleotides of a nucleic acid sequence that as set forth in any one of SEQ ID NOs: 151-300.

In some embodiments, the oligonucleotide comprises or consists of a nucleic acid sequence as set forth in any one of SEQ ID NOs: 1-150. In some embodiments, the oligonucleotide comprises a nucleic acid sequence as set forth in any one of SEQ ID NOs: 1-150, wherein one or more of the thymine nucleobases of the nucleic acid sequence are replaced with uracil nucleobases (e.g., one thymine is replaced with a uracil, two thymines are replaced with a uracil, or all of the thymines are replaced with uracils)

In some embodiments, at least one of the cytosines in the oligonucleotide is a 5-methylcytosine. In some embodiments, each of the cytosines in the oligonucleotide is a 5-methylcytosine.

Some aspects of the present disclosure provide a pharmaceutical composition comprising (i) any antisense oligonucleotide described herein; and (ii) a pharmaceutically acceptable carrier and/or excipient. A pharmaceutically acceptable carrier may be a solvent, aqueous buffer, liposome, polymeric micelle, nucleic acid nanostructure or nanoparticle. An excipient may be an antiadherent, binder, coating, or preservative.

Some aspects of the present disclosure provide a kit comprising a container housing any antisense oligonucleotide described herein. In some embodiments, the kit further comprises a container housing a pharmaceutically acceptable carrier and/or excipient. In some embodiments, the antisense oligonucleotide and the pharmaceutically acceptable carrier and/or excipient are housed in the same container.

Some aspects of the present disclosure provide methods of inhibiting expression of complement C5 in a cell (e.g., a mammalian cell, optionally a human cell, optionally a cell from a human subject having or suspected of having a disease or disorder associated with complement C5 dysfunction). In some embodiments, a method of inhibiting expression of complement C5 in a cell comprises contacting the cell with any antisense oligonucleotide described herein or a pharmaceutical composition (or formulation) described herein.

Yet further aspects of the present disclosure provide methods of inhibiting translation of a complement C5 pre-mRNA in a cell (e.g., a mammalian cell, optionally a human cell, optionally a cell from a human subject having or suspected of having a disease or disorder associated with complement C5 dysfunction). In some embodiments, a method of inhibiting translation of a complement C5 pre-mRNA in a cell comprises contacting the cell with any antisense oligonucleotide described herein or a pharmaceutical composition (or formulation) described herein.

Yet further aspects of the present disclosure provide methods of treating a disease or disorder associated with complement C5 dysfunction of complement C5 in a subject. In some embodiments, a method of treating a disease or disorder associated with complement C5 dysfunction of complement C5 in a subject comprises administering an effective amount of any antisense oligonucleotide described herein or a pharmaceutical composition (or formulation) described herein, thereby treating the subject.

In some embodiments, the disease or disorder associated with complement C5 dysfunction is a neurological disease, a primary dysregulation disorder, an autoimmune disease, an inflammatory disease, a degenerative disease, an ischaemia-reperfusion, or an acute injury. In some embodiments, the disease or disorder associated with complement C5 dysfunction is multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD). In some embodiments, the disease or disorder associated with complement C5 dysfunction is paroxysmal nocturnal hemoglobinuria (PNH) or atypical hemolytic uremic syndrome.

In some embodiments, the subject is a human subject. In some embodiments, the subject has elevated levels of complement C5 expression, relative to a healthy subject.

In some embodiments, the antisense oligonucleotide or the pharmaceutical composition is administered by intrathecal injection, intravenous injection, intramuscular injection, inhalation, subcutaneous injection, and/or intracranial injection.

In some embodiments, administration of the antisense oligonucleotide or the pharmaceutical composition results in decreased translation of a complement C5 pre-mRNA by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to translation of the pre-mRNA prior to administration.

In some embodiments, the complement C5 pre-mRNA comprises the nucleic acid sequence of SEQ ID NO: 301.

In some embodiments, administration of the antisense oligonucleotide or the pharmaceutical composition results in decreased expression of complement C5 by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to expression of complement C5 prior to administration.

### DETAILED DESCRIPTION

Described herein are compositions, methods, and kits for inhibition of Complement C5 gene expression. The inventors have discovered that targeting intronic sequences of a Complement C5 pre-mRNA using antisense oligonucleotides (e.g., gapmer antisense oligonucleotides) is effective for inhibition of Complement C5 gene expression. For example, compositions of antisense oligonucleotides that are complementary to (and bind to) introns of Complement C5 pre-mRNA (e.g., intron 1, 2, 3, 4, 5, 6, 7, 8, 9, 12, 14, 15, 16, 17, 18, and/or 20 of human Complement C5 pre-mRNA) may be useful for inhibition of transcription and/or translation of complement C5 expression. Furthermore, described herein are methods for treating a subject (e.g., a human patient) having a disease associated with complement C5 dysfunction (e.g., paroxysmal nocturnal hemoglobinuria (PNH) or atypical hemolytic uremic syndrome (aHUS)) by administering an antisense oligonucleotide that targets a Complement C5 pre-mRNA (e.g., an antisense oligonucleotide as described herein).

### Compositions of Antisense Oligonucleotides

Antisense oligonucleotides that are useful for targeting introns of Complement C5 pre-mRNA are typically complementary to a sequence within the desired intron. In some embodiments, an antisense oligonucleotide of the disclosure is complementary to an intron of a human complement C5 pre-mRNA. In some embodiments, an antisense oligonucleotide of the disclosure is complementary to an intron of a complement C5 pre-mRNA comprising the nucleic acid sequence of SEQ ID NO: 301. In some embodiments, an antisense oligonucleotide of the disclosure comprises a first sequence that is complementary to an intron of a complement C5 pre-mRNA and a second sequence that is complementary to an exon of a complement C5 pre-mRNA. Thus, in some embodiments, an antisense oligonucleotide is complementary to (and binds to) an intron-exon junction (or intron-exon boundary) of a complement C5 pre-mRNA. As used herein, the term “antisense oligonucleotide” generally refers to a means a polymer of linked nucleosides and/or nucleotides, wherein the polymer is capable of binding to (e.g., is complementary to) a target sequence, and wherein each of the nucleotides and/or nucleosides can be modified or unmodified, and the linkages between each of the nucleotides and/or nucleosides can be a modified or unmodified linkage.

An antisense oligonucleotide may be complementary to a sequence of any intron of a complement C5 pre-mRNA. In some embodiments, an antisense oligonucleotide is complementary to a sequence of intron 1, intron 2, intron 3, intron 4, intron 5, intron 6, intron 7, intron 8, intron 9, intron 12, intron 14, intron 15, intron 16, intron 17, intron 18, or intron 20 of human Complement C5 pre-mRNA (e.g., comprising the nucleic acid sequence of SEQ ID NO: 301). In some embodiments, a human Complement C5 gene is as described in RefSeqGene NG_007364.1. A Complement C5 gene or Complement C5 pre-mRNA may be a naturally occurring sequence variations of a wild-type (“normal”) Complement C5 gene or Complement C5 pre-mRNA, e.g., single nucleotide polymorphisms, e.g., as described in NCBI dbSNP Accession NOs: rs121909588 and rs121909587. As used herein, the term “target sequence” may refer to a segment or portion of a nucleic acid sequence of a Complement C5 pre-mRNA.

An antisense oligonucleotide may comprise a first sequence that is complementary to a sequence of any intron of a complement C5 pre-mRNA and a second sequence that is complementary to an exon of a complement C5 pre-mRNA. For example, in some embodiments, an antisense oligonucleotide comprises a first sequence that is complementary to intron 4 of a Complement C5 pre-mRNA and a second sequence that is complementary to exon 4 of the Complement C5 pre-mRNA. In some embodiments, an antisense oligonucleotide comprises a first sequence that is complementary to intron 12 of a Complement C5 pre-mRNA and a second sequence that is complementary to exon 12 of the Complement C5 pre-mRNA. in some embodiments, an antisense oligonucleotide comprises a first sequence that is complementary to intron 20 of a Complement C5 pre-mRNA and a second sequence that is complementary to exon 20 of the Complement C5 pre-mRNA.

In some embodiments, the antisense oligonucleotide modulates the splicing or any other processing of the complement C5 pre-mRNA. In some embodiments, the antisense oligonucleotide is designed to cause degradation of the pre-mRNA. In some embodiments, the antisense oligonucleotide is a gapmer, a mixmer, a small interfering RNA (siRNA), a ribozyme or an aptamer that modulates the splicing or any other processing, and/or causes degradation of the complement C5 pre-mRNA. In some embodiments, the antisense oligonucleotide is an RNAi oligonucleotide (e.g., siRNA, short hairpin RNA (shRNA)), microRNA, phosphorodiamidite morpholino, or a peptide nucleic acid. The antisense oligonucleotides of the disclosure may be single-stranded or double-stranded. In some embodiments, a double-stranded oligonucleotide comprises a first strand that is antisense to the target sequence and a second strand that is sense to the target sequence. In some embodiments, an antisense oligonucleotide may comprise one or more modified nucleotides (e.g. 2′-O-methyl sugar modified nucleotides such as 2′O-methoxyethyl RNA nucleotides). In some embodiments, an antisense oligonucleotide may comprise one or more modified internucleotide linkages (e.g., phosphorothioate linkages). In some embodiments, an antisense oligonucleotide may comprise one or more phosphorothioate linkages (e.g., in the Rp or Sp stereochemical conformations). In some embodiments, oligonucleotides in one format (e.g., gapmers) may be suitably adapted to another format (e.g., siRNA) by incorporating functional sequences and/or altering the sugar moiety (e.g., DNA to RNA, or vice versa) from one format to the other format. Any one of the antisense oligonucleotides of Table 1 (or any antisense oligonucleotide comprising at least 8, at least 10, or at least 12 or more contiguous nucleotides of any one of the antisense oligonucleotide sequences of Table 1) may comprise one or more modified nucleotides (e.g. 2′-O-methyl sugar modified nucleotides such as 2′O-methoxyethyl RNA nucleotides). In some embodiments, any one of the antisense oligonucleotides of Table 1 (or any antisense oligonucleotide comprising at least 8, at least 10, or at least 12 or more contiguous nucleotides of any one of the antisense oligonucleotide sequences of Table 1) comprises one or more modified internucleotide linkages (e.g., phosphorothioate linkages). In some embodiments, any one of the antisense oligonucleotides of Table 1 (or any antisense oligonucleotide comprising at least 8, at least 10, or at least 12 or more contiguous nucleotides of any one of the antisense oligonucleotide sequences of Table 1) comprises one or more phosphorothioate linkages (e.g., in the Rp or Sp stereochemical conformations).

In some embodiments, the entire length of the antisense oligonucleotide is complementary to an intron of a complement C5 pre-mRNA. In some embodiments, a region of the antisense oligonucleotide comprises a sequence that is complementary to an intron of a pre-mRNA. In some embodiments, a region of the antisense oligonucleotide that comprises a sequence complementary to an intron of a pre-mRNA is at least 10-15, 10-20, 15-25, 15-22, or about 20 nucleotides in length. As used herein, the term “region” with respect to the antisense oligonucleotide refers to a segment or portion of the antisense oligonucleotide. A region, segment, or portion of the antisense oligonucleotide may represent a specific number of contiguous nucleotides or nucleosides of the antisense oligonucleotide.

In some embodiments, the antisense oligonucleotide comprises a sequence that is complementary to an intron of a pre-mRNA and further comprises one or two sequences at the 5′ and/or 3′ end of the complementary sequence. Thus, in some embodiments, the antisense oligonucleotide comprises a sequence that is complementary to an intron of a pre-mRNA (e.g., a sequence that is at least 8 nucleotides in length) and additional nucleotides located upstream and/or downstream of that complementary sequence. In some embodiments, the antisense oligonucleotide comprises 1-15, 1-12, 1-10, 1-8, 1-6, 1-5, 1-3, 2-12, 4-8, or 3-10 nucleotides that are located at the 5′ and/or 3′ end of the complementary sequence. In some embodiments, the antisense oligonucleotide comprises 1-15, 1-12, 1-10, 1-8, 1-6, 1-5, 1-3, 2-12, 4-8, or 3-10 nucleotides that are located upstream and/or downstream of the complementary sequence. In some embodiments, the antisense oligonucleotide comprises 1-15, 1-12, 1-10, 1-8, 1-6, 1-5, 1-3, 2-12, 4-8, or 3-10 nucleotides (e.g., contiguous nucleotides) that are not complementary to a Complement C5 pre-mRNA.

In some embodiments, the antisense oligonucleotide comprises a nucleic acid sequence as set forth in any one of SEQ ID NOs: 1-150. In some embodiments, the antisense oligonucleotide comprises at least 10, at least 11, at least 12, at least 13, at least 14, at least 15, at least 16, at least 17, at least 18, at least 19, or at least 20 contiguous nucleotides of a nucleic acid sequence that as set forth in any one of SEQ ID NOs: 1-150. In some embodiments, the antisense oligonucleotide comprises at least 10, at least 11, at least 12, at least 13, at least 14, at least 15, at least 16, at least 17, at least 18, at least 19, or at least 20 contiguous nucleotides of a nucleic acid sequence that as set forth in any one of SEQ ID NOs: 1-150, wherein one or more thymines in the nucleic acid sequence with uracils.

In some embodiments, the antisense oligonucleotide consists of a nucleic acid sequence as set forth in any one of SEQ ID NOs: 1-150. In some embodiments, an antisense oligonucleotide comprises a region that is complementary to an intron of a pre-mRNA, wherein said region comprises at least 10, at least 11, at least 12, at least 13, at least 14, at least 15, at least 16, at least 17, at least 18, at least 19, or at least 20 contiguous nucleotides of a nucleic acid sequence as set forth in any one of SEQ ID NOs: 1-150. In some embodiments, the antisense oligonucleotide comprises at least 10, at least 11, at least 12, at least 13, at least 14, at least 15, at least 16, at least 17, at least 18, at least 19, or at least 20 contiguous nucleotides of a nucleic acid sequence as set forth in any one of SEQ ID NOs: 4, 11, 12, 34, 39, 47, 53, 55, 56, 57, 60, 62, 66, 69, 71, 77, 86, 99, 105, 108, 109, 110, 115, and 119.

In some embodiments, the antisense oligonucleotide comprises at least 10, at least 11, at least 12, at least 13, at least 14, at least 15, at least 16, at least 17, at least 18, at least 19, or at least 20 contiguous nucleotides of any one of the antisense oligonucleotide sequences of Table 1. In some embodiments, the antisense oligonucleotide comprises at least 10, at least 11, at least 12, at least 13, at least 14, at least 15, at least 16, at least 17, at least 18, at least 19, or at least 20 contiguous nucleotides that are complementary to any one of the target sequences of Table 1.

TABLE 1
Antisense oligonucleotides for targeting Complement C5 pre-mRNA

| | | | SEQ ID | Target Sequence |
|---|---|---|---|---|
| Antisense | SEQ ID | | NO of | Region in |
| Oligonucleotide | NO of | | Target | Complement C5 |
| (ASO) | ASO | Target Sequence | Sequence | gene (NG_007364.1) |
| gctggtctatgtgatggtag | 1 | ctaccatcacatagaccagc | 151 | Intron 1 |
| atcagaggctgtatcagcac | 2 | gtgctgatacagcctctgat | 152 | Intron 1 |
| cctctaacagaagaaccatg | 3 | catggttcttctgttagagg | 153 | Intron 3 |
| tggttcattgggaataggtc | 4 | gacctattcccaatgaacca | 154 | Intron 3 |
| caacctttgtgggcagagac | 5 | gtctctgcccacaaaggttg | 155 | Intron 3 |
| ggtcatacagctcacagcag | 6 | ctgctgtgagctgtatgacc | 156 | Intron 3 |
| gcccaagtcagaaagtcaca | 7 | tgtgactttctgacttgggc | 157 | Intron 3 |
| agtgtagtgtgaggacaggg | 8 | ccctgtcctcacactacact | 158 | Intron 4 |
| ttggactacacatgggggtc | 9 | gacccccatgtgtagtccaa | 159 | Intron 4 |
| gcaaggaacaagaccacacc | 10 | ggtgtggtcttgttccttgc | 160 | Intron 5 |
| gcttatgaagagccaggtga | 11 | tcacctggctcttcataagc | 161 | Intron 5 |
| gctcttcaggggactagatc | 12 | gatctagtcccctgaagagc | 162 | Intron 6 |
| ggtatagttggacaagctgt | 13 | acagcttgtccaactatacc | 163 | Intron 6 |
| agacatatggacaggacctt | 14 | aaggtcctgtccatatgtct | 164 | Intron 7 |
| ggcagctaacctagtgtaga | 15 | tctacactaggttagctgcc | 165 | Intron 8 |
| tttcaccaacaggcttcatc | 16 | gatgaagcctgttggtgaaa | 166 | Intron 12 |
| caacaccctaccctaaacct | 17 | aggtttagggtagggtgttg | 167 | Intron 14 |
| ggaatctcaagggatacctg | 18 | caggtatcccttgagattcc | 168 | Intron 16 |
| ggttaacttgaggggcagtc | 19 | gactgcccctcaagttaacc | 169 | Intron 17 |
| tagggcagggagaggaatac | 20 | gtattcctctccctgcccta | 170 | Intron 17 |
| gcagtggccagaaggtttac | 21 | gtaaaccttctggccactgc | 171 | Intron 18 |
| gtagccaagatcagtgtgag | 22 | ctcacactgatcttggctac | 172 | Intron 20 |
| gtggaagcaaaatatttaaa | 23 | tttaaatattttgcttccac | 173 | Intron 5 |
| cagaaactatagaacaggct | 24 | agcctgttctatagtttctg | 174 | Intron 2 |
| tctctggacccttagtatta | 25 | taatactaagggtccagaga | 175 | Intron 2 |
| gcaattgaaagtctctttgg | 26 | ccaaagagactttcaattgc | 176 | Intron 2 |
| gaattgagagtcttttgagc | 27 | gctcaaaagactctcaattc | 177 | Intron 3 |
| tggacaaagactgatgaccc | 28 | gggtcatcagtctttgtcca | 178 | Intron 3 |
| agggtcatacagctcacagc | 29 | gctgtgagctgtatgaccct | 179 | Intron 3 |
| gcttggctctatcttaagtg | 30 | cacttaagatagagccaagc | 180 | Intron 3 |
| gtagtaggtgatgaagctgg | 31 | ccagcttcatcacctactac | 181 | Intron 3 |
| gaggtcagtttggcttgtgt | 32 | acacaagccaaactgacctc | 182 | Intron 3 |
| gttaacaaggcaaaggaggg | 33 | ccctcctttgccttgttaac | 183 | Intron 3 |
| ggctagtttttaacccagtt | 34 | aactgggttaaaaactagcc | 184 | Intron 3 |
| ggcaccataaacccagtatc | 35 | gatactgggtttatggtgcc | 185 | Intron 3 |
| taactcacagctgtttgaga | 36 | tctcaaacagctgtgagtta | 186 | Intron 3 |
| ttgggagcttgattcagaca | 37 | tgtctgaatcaagctcccaa | 187 | Intron 3 |
| gatttagtgtagtgtgagga | 38 | tcctcacactacactaaatc | 188 | Intron 4 |
| tcttagtgattggtgggtgg | 39 | ccacccaccaatcactaaga | 189 | Intron 4 |
| ggaccattagtagtatacct | 40 | aggtatactactaatggtcc | 190 | Intron 4 |
| tctgagctcatggtatttgt | 41 | acaaataccatgagctcaga | 191 | Intron 4 |
| ctcacctctcattggtcttc | 42 | gaagaccaatgagaggtgag | 192 | Intron 4 |
| tacatggatggtcttcacta | 43 | tagtgaagaccatccatgta | 193 | Intron 4 |
| agggcatttgagctgacatt | 44 | aatgtcagctcaaatgccct | 194 | Intron 4 |
| catagcaggcaacataagtg | 45 | cacttatgttgcctgctatg | 195 | Intron 4 |
| ttctttggactacacatggg | 46 | cccatgtgtagtccaaagaa | 196 | Intron 4 |
| tggccccctttaactggtta | 47 | taaccagttaaagggggcca | 197 | Intron 5 |
| acaagaccacaccagccttt | 48 | aaaggctggtgtggtcttgt | 198 | Intron 5 |
| agcaaccagagcaaggaaca | 49 | tgttccttgctctggttgct | 199 | Intron 5 |
| gttggagaaatgatagcagt | 50 | actgctatcatttctccaac | 200 | Intron 5 |
| gagccaggtgaagcccattc | 51 | gaatgggcttcacctggctc | 201 | Intron 5 |
| agcttctactctgggtcata | 52 | tatgacccagagtagaagct | 202 | Intron 5 |
| gggcctattatgttgaactg | 53 | cagttcaacataataggccc | 203 | Intron 5 |
| ggttggaaacaaagcatggt | 54 | accatgctttgtttccaacc | 204 | Intron 5 |
| gccagtacttctagccatac | 55 | gtatggctagaagtactggc | 205 | Intron 6 |
| ctagtcaagctcttcagggg | 56 | cccctgaagagcttgactag | 206 | Intron 6 |
| ggggtaaggaaacctaattg | 57 | caattaggtttccttacccc | 207 | Intron 6 |
| gcctaggctcagggggactt | 58 | aagtccccctgagcctaggc | 208 | Intron 7 |
| ggagtgattaatacaaaggg | 59 | ccctttgtattaatcactcc | 209 | Intron 8 |
| aacctagtgtagaagagtca | 60 | tgactcttctacactaggtt | 210 | Intron 8 |
| caacccctcagcattattga | 61 | tcaataatgctgaggggttg | 211 | Intron 8 |
| tgtctctgggcatataaggg | 62 | cccttatatgcccagagaca | 212 | Intron 8 |
| gattaagcttcaccatctac | 63 | gtagatggtgaagcttaatc | 213 | Intron 9 |
| gtgttcacagagcatagata | 64 | tatctatgctctgtgaacac | 214 | Intron 9 |
| gaaggttttgagtactacac | 65 | gtgtagtactcaaaaccttc | 215 | Intron 15 |
| taggttaacttgaggggcag | 66 | ctgcccctcaagttaaccta | 216 | Intron 17 |
| gcctgatgattttgaagact | 67 | agtcttcaaaatcatcaggc | 217 | Intron 17 |
| cagaaggtttactagaggac | 68 | gtcctctagtaaaccttctg | 218 | Intron 18 |
| ggccaagtaaaatttgtgtg | 69 | cacacaaattttacttggcc | 219 | Intron 20 |
| gtggcatgattcataggatt | 70 | aatcctatgaatcatgccac | 220 | Intron 20 |
| ggtctgactgtcttagtatc | 71 | gatactaagacagtcagacc | 221 | Intron 20 |
| acagttgagcagagtttggc | 72 | gccaaactctgctcaactgt | 222 | Intron 20 |
| gagtgatcttagaaatacta | 73 | tagtatttctaagatcactc | 223 | Intron 1 |
| gttatttgatcaaggttgtt | 74 | aacaaccttgatcaaataac | 224 | Intron 2 |
| tgtctctaagcaatctatac | 75 | gtatagattgcttagagaca | 225 | Intron 2 |
| gggatgaaagttaattacaa | 76 | ttgtaattaactttcatccc | 226 | Intron 2 |
| catatatttctgtgatctag | 77 | ctagatcacagaaatatatg | 227 | Intron 3 |
| tacatcatcttacagtaaag | 78 | ctttactgtaagatgatgta | 228 | Intron 3 |
| ttgggcaatattctaaagtc | 79 | gactttagaatattgcccaa | 229 | Intron 3 |
| ttacacaggccttaatatgg | 80 | ccatattaaggcctgtgtaa | 230 | Intron 3 |
| aaggaggggcaagaatgtac | 81 | gtacattcttgcccctcctt | 231 | Intron 3 |
| gtaaaattctagggtgtgaa | 82 | ttcacaccctagaattttac | 232 | Intron 3 |
| gacatggacatcttttaatg | 83 | cattaaaagatgtccatgtc | 233 | Intron 3 |
| aaaagatatggcttattggc | 84 | gccaataagccatatctttt | 234 | Intron 3 |
| gcatagcctgacacatacta | 85 | tagtatgtgtcaggctatgc | 235 | Intron 3 |
| ttacatctgtaattaggcca | 86 | tggcctaattacagatgtaa | 236 | Intron 3 |
| tagtggatttaaagtacagt | 87 | actgtactttaaatccacta | 237 | Intron 3 |
| ttgtatgagcctttcacata | 88 | tatgtgaaaggctcatacaa | 238 | Intron 4 |
| gcaagattgtacttaaatgg | 89 | ccatttaagtacaatcttgc | 239 | Intron 4 |
| gtctgttacatatgtcatgt | 90 | acatgacatatgtaacagac | 240 | Intron 4 |
| ctgatagaaaaagggcattt | 91 | aaatgccctttttctatcag | 241 | Intron 4 |
| acaagaattatacttgtagc | 92 | gctacaagtataattcttgt | 242 | Intron 4 |
| ccatgactgaaattcacagt | 93 | actgtgaatttcagtcatgg | 243 | Intron 4 |
| aatatggttcatttcagcca | 94 | tggctgaaatgaaccatatt | 244 | Intron 4 |
| tgagatagggagtaaaacaa | 95 | ttgttttactccctatctca | 245 | Intron 5 |
| acattgagataagtgtcatg | 96 | catgacacttatctcaatgt | 246 | Intron 5 |
| ttataagctagtggggaata | 97 | tattccccactagcttataa | 247 | Intron 5 |
| atacaagacaagcttataca | 98 | tgtataagcttgtcttgtat | 248 | Intron 5 |
| tgaactcatatcatctgtgg | 99 | ccacagatgatatgagttca | 249 | Intron 8 |
| gcttagagctcatatggaat | 100 | attccatatgagctctaagc | 250 | Intron 9 |
| ataggaacttatgagaattg | 101 | caattctcataagttcctat | 251 | Intron 14 |
| gttaaatcaagaactaagtc | 102 | gacttagttcttgatttaac | 252 | Intron 14 |
| aggtactaattgttacatgg | 103 | ccatgtaacaattagtacct | 253 | Intron 14 |
| acttgtaaatagatctagtc | 104 | gactagatctatttacaagt | 254 | Intron 15 |
| ttaactctggcctagtgtac | 105 | gtacactaggccagagttaa | 255 | Intron 17 |
| cctgttggtacactaaattt | 106 | aaatttagtgtaccaacagg | 256 | Intron 17 |
| ccagatgattatagttgaaa | 107 | tttcaactataatcatctgg | 257 | Intron 17 |
| tatggccattaatctgtaac | 108 | gttacagattaatggccata | 258 | Intron 17 |
| ctagctaatgggtaatgtca | 109 | tgacattacccattagctag | 259 | Intron 18 |
| acagctattgtctaccaact | 110 | agttggtagacaatagctgt | 260 | Intron 18 |
| atattctaaaatagggaagc | 111 | gcttccctattttagaatat | 261 | Intron 18 |
| ctctatgatgagaattcaag | 112 | cttgaattctcatcatagag | 262 | Intron 18 |
| acccaacttaacagagacat | 113 | atgtctctgttaagttgggt | 263 | Intron 18 |
| gaatattgattaacccaact | 114 | agttgggttaatcaatattc | 264 | Intron 18 |
| aatctggttaatgagttagg | 115 | cctaactcattaaccagatt | 265 | Intron 20 |
| cataattttagtcagtgtgg | 116 | ccacactgactaaaattatg | 266 | Intron 20 |
| ttatcaatgaggactgattt | 117 | aaatcagtcctcattgataa | 267 | Intron 20 |
| caaaccctgtgagtaaattc | 118 | gaatttactcacagggtttg | 268 | Intron 20 |
| gtctgttttgatgagatggt | 119 | accatctcatcaaaacagac | 269 | Intron 20 |
| tatatgtctaagactaggtg | 120 | cacctagtcttagacatata | 270 | Intron 20 |
| ttaacccagtttgtgtttag | 121 | ctaaacacaaactgggttaa | 271 | Intron 3 |
| ttttaacccagtttgtgttt | 122 | aaacacaaactgggttaaaa | 272 | Intron 3 |
| gtttttaacccagtttgtgt | 123 | acacaaactgggttaaaaac | 273 | Intron 3 |
| tagtttttaacccagtttgt | 124 | acaaactgggttaaaaacta | 274 | Intron 3 |
| ttggctagtttttaacccag | 125 | tgggttaaaaactagccaaa | 275 | Intron 3 |
| tatttggctagtttttaacc | 126 | ggttaaaaactagccaaata | 276 | Intron 3 |
| ctatttggctagtttttaac | 127 | gttaaaaactagccaaatag | 277 | Intron 3 |
| tactatttggctagttttta | 128 | taaaaactagccaaatagta | 278 | Intron 3 |
| ataagggaaatgattcagac | 129 | gtctgaatcatttcccttat | 279 | Intron 8 |
| gcatataagggaaatgattc | 130 | gaatcatttcccttatatgc | 280 | Intron 8 |
| gggcatataagggaaatgat | 131 | atcatttcccttatatgccc | 281 | Intron 8 |
| ctgggcatataagggaaatg | 132 | catttcccttatatgcccag | 282 | Intron 8 |
| tctctgggcatataagggaa | 133 | ttcccttatatgcccagaga | 283 | Intron 8 |
| gctgtctctgggcatataag | 134 | cttatatgcccagagacagc | 284 | Intron 8 |
| tatgctgtctctgggcatat | 135 | atatgcccagagacagcata | 285 | Intron 8 |
| actatgctgtctctgggcat | 136 | atgcccagagacagcatagt | 286 | Intron 8 |
| aagtaaaatttgtgtgtgca | 137 | tgcacacacaaattttactt | 287 | Intron 20 |
| ccaagtaaaatttgtgtgtg | 138 | cacacacaaattttacttgg | 288 | Intron 20 |
| gtggccaagtaaaatttgtg | 139 | cacaaattttacttggccac | 289 | Intron 20 |
| cctgtggccaagtaaaattt | 140 | aaattttacttggccacagg | 290 | Intron 20 |
| catcctgtggccaagtaaaa | 141 | ttttacttggccacaggatg | 291 | Intron 20 |
| ttacatcctgtggccaagta | 142 | tacttggccacaggatgtaa | 292 | Intron 20 |
| atttacatcctgtggccaag | 143 | cttggccacaggatgtaaat | 293 | Intron 20 |
| cccatttacatcctgtggcc | 144 | ggccacaggatgtaaatggg | 294 | Intron 20 |
| actatgaaagttaagacagt | 145 | actgtcttaactttcatagt | 295 | Exon 4 and intron 4 |
| tacttactatgaaagttaag | 146 | cttaactttcatagtaagta | 296 | Exon 4 and intron 4 |
| cttaccaagtaattatagtg | 147 | cactataattacttggtaag | 297 | Exon 12 and intron 12 |
| catataccttcagcccaaag | 148 | ctttgggctgaaggtatatg | 298 | Exon 20 and Intron 20 |
| gcaacacatataccttcagc | 149 | gctgaaggtatatgtgttgc | 299 | Exon 20 and Intron 20 |
| gtatcagcaacacatatacc | 150 | ggtatatgtgttgctgatac | 300 | Exon 20 and Intron 20 |

In some embodiments, the antisense oligonucleotide is complementary to at least 10, at least 11, at least 12, at least 13, at least 14, at least 15, at least 16, at least 17, at least 18, at least 19, or at least 20 contiguous nucleotides of a nucleic acid sequence as set forth in any one of 151-300. In some embodiments, the antisense oligonucleotide comprises a region (e.g., a region of at least 8, 9, 10, 12, or 15 contiguous nucleotides) that is complementary to a nucleic acid sequence that as set forth in any one of 151-300. An antisense oligonucleotide comprising a region that is complementary to a nucleic acid sequence that as set forth in any one of 151-300 may be a gapmer, a mixmer, a ribozyme, an aptamer, an RNAi oligonucleotide (e.g., siRNA, shRNA), a microRNA, a phosphorodiamidite morpholino, or a peptide nucleic acid.

In some embodiments, the antisense oligonucleotide comprises 12-50 linked nucleotides (optionally 15-30 linked nucleotides) and having a nucleobase sequence comprising at least 8, 9, 10, 11, 12, 13, 14, 15, 16, 17, 18, 19, or 20 consecutive nucleobases of any of the nucleobase sequences of SEQ ID NOs: 1-150.

As used herein, the term “complementary” refers to the ability for a pair of nucleotides or a pair of nucleic acid sequences (e.g., an antisense oligonucleotide of the disclosure and a target pre-mRNA sequence) to bind to one another (e.g. using hydrogen bond pairing between two nucleotides). Two nucleic acid sequences or nucleic acid strands are “complementary” to one another if they base-pair, or bind, to each other to form a double-stranded nucleic acid molecule via Watson-Crick interactions and non-Watson-Crick base pairing (also referred to as hybridization). Binding to form a double-stranded nucleic acid molecule refers to an association between at least two strands due to, for example, electrostatic, hydrophobic, ionic and/or hydrogen-bond interactions (e.g., under physiological conditions). Non-Watson-Crick base pairing include wobble base pairing and Hoogsteen base pairing. In some embodiments, for complementary base pairings, adenosine-type bases (A) are complementary to thymidine-type bases (T) or uracil-type bases (U); cytosine-type bases (C) are complementary to guanosine-type bases (G); and universal bases such as 3-nitropyrrole, 5-nitroindole, or inosine (I) can hybridize to and are considered complementary to any A-type, C-type, U-type, G-type, or T-type bases. In some embodiments, two nucleic acid sequences are complementary to one another if at least 70%, 75%, 80%, 85%, 90%, 95%, 99%, or 100% of the nucleobases across the length of one of the sequences are base-paired to nucleobases of the other sequence. Thus, in some embodiments, a sequence within an antisense oligonucleotide is complementary to a target sequence (e.g., an intron of a complement C5 pre-mRNA) if at least 70%, 75%, 80%, 85%, 90%, 95%, 99%, or 100% of the nucleobases across the length of said sequence are base-paired to nucleobases of the target sequence. In some embodiments, an antisense oligonucleotide comprises a sequence that is sufficiently complementary to a cognate target nucleotide sequence, (e.g., a target sequence of a complement C5 pre-mRNA), such that the antisense oligonucleotide is capable of hybridizing to the cognate target nucleotide sequence (e.g., under physiological conditions such as the conditions in a cell). In some embodiments, an antisense oligonucleotide comprises a sequence that contains 1, 2, 3, 4, or 5 nucleobase mismatches with its cognate target nucleotide sequence. For example, an antisense oligonucleotide may comprise a sequence of 15-25 nucleotides that contains 1, 2, 3, 4, or 5 nucleobase mismatches with its cognate target nucleotide sequence.

Antisense oligonucleotides can be any length necessary for inhibition of complement C5 gene expression, which can vary depending on the format of the oligonucleotide. In some embodiments, an oligonucleotide is 10-200 nucleotides in length. In some embodiments, an oligonucleotide is 10-100, 100-200, 50-150, 10-50, 10-30, 15-30, 15-25, or 15-60 nucleotides in length. In some embodiments, an oligonucleotide is 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, or more nucleotides in length. In some embodiments, an oligonucleotide comprises a sequence that is complementary to a complement C5 pre-mRNA, wherein said sequence is 7, 8, 9, 10, 11, 12, 13, 14, 15, 16, 17, 18, 19, 20, 21, 22, 23, 24, 25, 26, 27, 28, 29, 30, 31, 32, 33, 34, 35, or 40 nucleotides in length.

In some embodiments, an antisense oligonucleotide is modified to comprise a modified sugar moiety, a modified internucleotide linkage, a modified nucleobase and/or combinations thereof. An antisense oligonucleotide can be modified in any manner that will cause the oligonucleotide to have decreased immune stimulatory activity, increased resistance to degradation (e.g., in a cell, e.g., resulting from nuclease activity), decreased toxicity (e.g., to cells or subjects), increased cellular uptake, increased solubility properties, decreased side effects, and/or decreased off-target effects.

In some embodiments, an antisense oligonucleotide comprises one or more phosphodiester internucleotide linkages. In some embodiments, an antisense oligonucleotide is modified to comprise one or more modified internucleotide linkages. A modified internucleotide linkage may be any internucleotide linkage other than a phosphodiester internucleoside linkage. Examples of modified internucleotide linkages include phosphorothioates (e.g., having Rp or Sp stereochemistry), phosphotriesters, methyl phosphonates, short chain alkyl linkages, cycloalkyl linkages, and heteroatomic or heterocyclic linkages. Additional phosphorus-containing linkages that may be used include phosphorodithioates, aminoalkylphosphotriesters, phosphinates, phosphoramidates comprising 3′-amino phosphoramidate and aminoalkylphosphoramidates, thionophosphoramidates, thionoalkylphosphonates, thionoalkylphosphotriesters, and boranophosphates. In some embodiments, at least one, two, three, four, five or more of the internucleotide linkages of an oligonucleotide are modified internucleotide linkages. In some embodiments, at least 10%, 20%, 30%, 40%, 50%, 60%, 70%, 80%, 90%, or 95% of the internucleotide linkages of an oligonucleotide are modified internucleotide linkages.

In some embodiments, an antisense oligonucleotide is modified to comprise one or more modified nucleotides comprising modified sugar moieties. In some embodiments, an oligonucleotide comprises a 2′-modified nucleotide such as a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA) nucleotide. In some embodiments, an ‘oligonucleot’ de comprises at least one nucleotide having a modification. In some embodiments, at least 10%, 20%, 30%, 40%, 50%, 60%, 70%, 80%, 90%, or 95% of the nucleotides within an oligonucleotide comprise modification. In some embodiments, all of the nucleotides within an oligonucleotide comprise a modification. In some embodiments, an oligonucleotide comprises at least one nucleotide having a 2′-O-methyl modification. In some embodiments, at least 10%, 20%, 30%, 40%, 50%, 60%, 70%, 80%, 90%, or 95% of the nucleotides within an oligonucleotide comprise a 2′-O-methyl modification (e.g., 2′-MOE modification). In some embodiments, all of the nucleotides within an oligonucleotide comprise a 2′-O-methyl modification. In other preferred embodiments, RNA modifications include 2′-fluoro, 2′-amino and 2′ O-methyl modifications on the ribose of pyrimidines, abasic residues or an inverted base at the 3′ end of the RNA. In some embodiments, an oligonucleotide comprises modified nucleotides in which the sugar moiety comprises a bridging linker connecting two atoms in the ring (e.g., connecting the 2′-O atom to the 4′-C atom of the sugar). In some embodiments, the oligonucleotides are locked nucleic acids (LNAs) comprising modified nucleotides in which the sugar moiety is ‘locked’ by a methylene bridge, an ethylene bridge, or any other linkage connecting the 2′-O atom and the 4′-C atom.

The oligonucleotide may comprise alternating nucleotides of different kinds. For example, an oligonucleotide may comprise alternating deoxyribonucleotides or ribonucleotides. An oligonucleotide may comprise alternating nucleotides having different sugar and/or internucleotide linkage modifications.

In some embodiments, the antisense oligonucleotide is a gapmer. A gapmer oligonucleotide generally has the formula 5′-X-Y-Z-3′, with X and Z as flanking segments around a central, gap segment Y. In some embodiments, the Y segment is a contiguous stretch of nucleotides (e.g., a stretch of 5-20 DNA nucleotides) that is capable of recruiting an RNAse, such as RNAse H. In some embodiments, the gapmer binds to the target pre-mRNA, at which point an RNAse is recruited and can then cleave the target pre-mRNA. In some embodiments, the X and Z regions are contiguous stretches of RNA nucleotides (e.g., 2-15 RNA nucleotides). In some embodiments, the X region and/or the Z region comprise modified nucleotides (e.g., 2-O-methoxyethyl (2′-MOE)).

In some embodiments, the central segment of the gapmer contains modified nucleotides known to be acceptable for efficient Rnase H action in addition to DNA nucleotides, such as C4′-substituted nucleotides, acyclic nucleotides, and arabino-configured nucleotides. In some embodiments, the central segment comprises one or more unmodified internucleotide linkages. In some embodiments, one or both flanking segment each independently comprise one or more modified internucleotide linkages (e.g., phosphorothioate internucleotide linkages). In some embodiments, the central segment and two flanking segments each independently comprise modified internucleotide linkages (e.g., phosphorothioate internucleotide linkages). In some embodiments, the majority of linkages (e.g., more than 50%, at least 75%, 80%, 90%) between individual nucleotides of the central segment and/or the flanking segments are modified internucleotide linkages. In some embodiments, the majority of linkages between individual nucleotides of the antisense oligonucleotide are modified internucleotide linkages. In some embodiments, each of the linkages between individual nucleotides of the antisense oligonucleotide are modified internucleotide linkages (e.g., phosphorothioate internucleotide linkages).

In some embodiments, the gapmer comprises a central segment (Y segment) of 5-20 nucleotides. In some embodiments, the gapmer comprises a central segment of 5-20 deoxyribonucleotides. In some embodiments, the central segment has a length of 5, 6, 7, 8, 9, 10, 11, 12, 13, 14, 15, 16, 17, 18, 19, or 20 nucleotides (e.g., deoxyribonucleotides).

The flanking segments (X and Z segments) may comprise a length of 1-20 nucleotides. The flanking segments can be the same length (i.e., X segment and Z segment consist of same number of nucleotides) or be different lengths. In some embodiments, each flanking segment can independently comprise a length of 2-15 nucleotides. In some embodiments, each flanking segment can independently comprise a length of 2-15 ribonucleotides. In some embodiments, each flanking segment can independently comprise a length of 2, 3, 4, 5, 6, 7, 8, 9, 10, 11, 12, 13, 14, or 15 nucleotides (e.g., ribonucleotides). In some embodiments, the gapmer comprises a central segment of 10 deoxyribonucleotides flanked by 5 ribonucleotides on either side of the central segment.

In some embodiments, an antisense oligonucleotide of the disclosure is attached (e.g., covalently attached) to a cell-targeting agent (e.g., an agent that enables the antisense oligonucleotide to be delivered to a specific cell target. A cell-targeting agent may include a protein (e.g., an antibody or antigen-binding fragment), a carbohydrate, a cell surface receptor ligand, a hormone, a cytokine, or a lipophilic polymer. In some embodiments, a cell-targeting agent is an agent that directs the delivery of an antisense oligonucleotide to neuronal cells, microglia, oligodendrocytes, astrocytes, and/or ependymal cells.

### Formulations

The antisense oligonucleotides of the disclosure may be formulated in any manner that enables binding, association, and/or inhibition of complement C5 pre-mRNA. In some embodiments, the oligonucleotides are formulated in a saline or buffer solution, for example. Further, in some embodiments, the antisense oligonucleotides are formulated as a pharmaceutical composition (e.g., for delivery to a subject). In some embodiments, pharmaceutical compositions comprise an antisense oligonucleotide as described herein, and a pharmaceutically acceptable carrier (e.g., a solvent, aqueous buffer, liposome, polymeric micelle, nucleic acid nanostructure or nanoparticle) and/or excipient (an antiadherent, binder, coating, or preservative).

In some embodiments, the antisense oligonucleotides are formulated in water or in a buffered solution. In some embodiments, a buffered aqueous solution is phosphate-buffered saline or an equivalent. In some embodiments, a buffered aqueous solution comprises sodium citrate, sodium phosphate, a tris base, a Good's buffering agent, or sodium hydroxide.

In some embodiments, the antisense oligonucleotides are dialyzed into water or in a buffered solution. In some embodiments, the antisense oligonucleotides are dialyzed into phosphate-buffered saline or an equivalent. In some embodiments, the antisense oligonucleotides are dialyzed into sodium citrate, sodium phosphate, a tris base, a Good's buffering agent, or sodium hydroxide.

In some embodiments, the antisense oligonucleotides are formulated for lyophilization. Such lyophilized formulations can subsequently be stored (e.g., at room temperature) for extended periods of time prior to being re-formulated as a solution (e.g., and administration to a subject).

In some embodiments, the antisense oligonucleotides are formulated as a pharmaceutical composition compatible with the intended route of administration of the oligonucleotide. Examples of routes of administration include parenteral, e.g., intrathecal, intravenous, intradermal, subcutaneous, administration. Typically, the route of administration is intrathecal, intravenous, intrathecal or subcutaneous. In certain circumstances, it will be desirable to administer the oligonucleotide 19ntraocularly, subretinally, subcutaneously, intraopancreatically, intranasally, parenterally, intravenously, intramuscularly, intrathecally, orally, intraperitoneally, or by inhalation. In such circumstances, the oligonucleotide will be formulated accordingly.

For administration of an injectable aqueous solution, a formulation comprising the oligonucleotide may be suitably buffered, if necessary, and the liquid diluent first rendered isotonic with sufficient saline or glucose. These particular aqueous solutions are especially suitable for intravenous, intrathecal, intramuscular, subcutaneous and intraperitoneal administration. In this connection, a sterile aqueous medium that can be employed will be known to those of skill in the art. Sterile injectable solutions are prepared by incorporating the oligonucleotide in a required amount necessary for efficacy in the appropriate solvent with various of the other ingredients enumerated herein, as required, followed by filtered sterilization.

The antisense oligonucleotides disclosed herein may also be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Delivery vehicles such as liposomes, nanocapsules, microparticles, microspheres, lipid particles, and vesicles may be used for the introduction of the compositions of the disclosure into suitable host cells. The formation and use of delivery vehicles such as liposomes is generally known to those of skill in the art.

The formulations described herein may contain at least about 0.1% of the antisense oligonucleotide or more, although the percentage of the oligonucleotide may, of course, be varied and may conveniently be between about 1 or 2% and about 70% or 80% or more of the weight or volume of the total formulation. The amount of oligonucleotide may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

### Methods of Administering the Antisense Oligonucleotides

The antisense oligonucleotides of the disclosure (or pharmaceutical compositions comprising an antisense oligonucleotide) may be administered to a cell or a subject (e.g., a human subject) in order to inhibit expression of complement C5 (e.g., inhibit complement C5 gene expression and/or complement C5 protein expression). In some embodiments, the antisense oligonucleotides described herein inhibit translation of a complement C5 pre-mRNA (e.g., by modulating splicing and/or any other processing of the pre-mRNA) when administered to a cell or subject. The oligonucleotide functions, in some embodiments, by binding (e.g., selectively binding) to an intron of the pre-mRNA and inhibiting (e.g., selectively inhibiting) translation of the complement C5 pre-mRNA. In this context, selectivity of binding (and inhibition) means that, in some embodiments, the oligonucleotides bind to the target intron of the complement C5 pre-mRNA preferentially relative to other sequences in the cell or subject (e.g., low levels of off-target effects). In some embodiments, the oligonucleotides bind to the target intron of a complement C5 pre-mRNA with more than 10%, 20%, 30%, 40%, 50%, 60%, 70%, 80%, 90%, or 95% binding affinity relative to any non-target sequences in the cell or subject.

A subject may be a human subject, non-human primate, a veterinary or agricultural animal (e.g., a cow, horse, pig, goat, sheep, etc.), a rodent (e.g., a mouse or rat), or a bird. In some embodiments, a subject may have or be at risk of having a disease or disorder associated with dysfunction of complement C5 (e.g., multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD), paroxysmal nocturnal hemoglobinuria (PNH)).

Further, in some embodiments, the disclosure provides methods of treating a disease or disorder associated with dysfunction of complement C5 in a subject (e.g., a human subject). The oligonucleotides of the disclosure can be administered to a subject in an amount effective to treat the subject. In some embodiments, the oligonucleotides of the disclosure can be administered to a subject in an amount effective to treat one or more symptoms of a disease or disorder associated with dysfunction of complement C5 (e.g., multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD), paroxysmal nocturnal hemoglobinuria (PNH)). Examples of symptoms of a disease or disorder associated with dysfunction of complement C5 include, but are not limited to, hemolysis, chronic inflammation, and elevated levels of complement C5. A person skilled in the art understands how to determine an amount or concentration of the antisense oligonucleotide that is necessary to be effective in treating the subject. The amount or concentration of the antisense oligonucleotide can vary depending on the severity of the disease being treated, off-target or side effects, the life circumstances of the subject, the duration of the treatment, and the route of administration (e.g., intrathecal administration), for example. In some embodiments, an effective amount or concentration is the maximum concentration of the oligonucleotide that is considered to be safe for the patient. In some embodiments, an effective amount or concentration will be the lowest possible concentration of the oligonucleotide that provides maximum inhibition.

In some embodiments, methods of administration of an antisense oligonucleotide to a cell involves contacting the cell with a suitable formulation of the oligonucleotide. For example, in some embodiments, the oligonucleotide is formulated for delivery to a cell in a lipid particle, a liposome, a vesicle, a nanosphere, or a nanoparticle. In some embodiments, the oligonucleotide contacted with the cell as a naked oligonucleotide (e.g., formulated in a buffered solution without any additional carriers or excipients). In some embodiments, the oligonucleotide is transfected into the cell (e.g., using a transfection reagent such as Lipofectamine). The cell may be a mammalian cell. In some embodiments, the cell is a human cell, optionally a cell from a human subject having or suspected of having a disease or disorder associated with complement C5 dysfunction.

In some embodiments, methods of administration of an antisense oligonucleotide to a subject involves administration by intrathecal injection, intravenous injection, intramuscular injection, inhalation, subcutaneous injection, or intracranial injection. In such methods, the oligonucleotide is formulated in accordance with its mode of administration. In some embodiments, methods of administration of an antisense oligonucleotide to a subject involve direct delivery into the brain parenchyma, e.g., by infusion into the brain, such as by continuous pump infusion. The oligonucleotide can be administered as a bolus injection or a continuous infusion over a period of time. In some embodiments, the subject is a mammal. In some embodiments, the subject is a human subject (e.g., a human having a disease or disorder associated with complement C5 dysfunction). In some embodiments, a disease or disorder associated with complement C5 dysfunction is a neurological disease, a primary dysregulation disorder, an autoimmune disease, an inflammatory disease, a degenerative disease, an ischaemia-reperfusion, or an acute injury. The disease or disorder associated with complement C5 dysfunction may be multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD).

In some embodiments, administration of an antisense oligonucleotide to a subject (e.g., human subject) treats the subject having a disease or disorder associated with complement C5 dysfunction. In some embodiments, the subject is a human subject (e.g., a human having a disease or disorder associated with complement C5 dysfunction). In some embodiments, a disease or disorder associated with complement C5 dysfunction is a neurological disease, a primary dysregulation disorder, an autoimmune disease, an inflammatory disease, a degenerative disease, an ischaemia-reperfusion, or an acute injury. The disease or disorder associated with complement C5 dysfunction may be multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD). “Treatment” or “treating” a subject generally involves administration of an antisense oligonucleotide described herein, resulting in curing, healing, alleviating, relieving, or ameliorating the disease or disorder, or symptoms of the disease or disorder. In some embodiments, methods of treatment can be useful in preventing onset of a disease or slowing the progression of a disease. Thus, the antisense oligonucleotides of the disclosure can be used prophylactically.

A subject in need of treatment (e.g., a subject having multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD)) may have elevated levels of complement C5 expression (e.g., expression of complement C5 mRNA and/or complement C5 protein) relative to a healthy subject. For example, in some embodiments, a subject in need of treatment has at least 5%, 10%, 25%, 40%, 50%, or 75% higher levels of complement C5 expression relative to a healthy subject. In some embodiments, complement C5 expression levels are elevated in cells of the nervous system and/or brain of a subject in need of treatment. In some embodiments, complement C5 expression levels are elevated in cerebrospinal fluid, serum, and/or blood of a subject in need of treatment. In some embodiments, complement C5 expression levels are elevated in neuronal cells, microglia, oligodendrocytes, astrocytes, and/or ependymal cells of a subject in need of treatment.

The antisense oligonucleotides (or pharmaceutical compositions comprising an antisense oligonucleotide) may be administered in dosages sufficient to inhibit expression of a Complement C5 gene or protein. For example, the antisense oligonucleotides may be administered at a dosage in the range of about 0.001 to about 200.0 milligrams per kilogram body weight of the subject per day. In some embodiments, the antisense oligonucleotides are administered at a dosage in the range of about 1 to 50 mg per kilogram body weight per day. In some embodiments, the dsRNA is administered at a dosage in the range of 2 to 50 mg/kg, 5 to 50 mg/kg, 10 to 50 mg/kg, 20 to 50 mg/kg. 2 to 10 mg/kg, 5 to 10 mg/kg, 10 to 20 mg/kg, or 15 to 30 mg/kg. The antisense oligonucleotides can be administered to a subject once or multiple times. In some embodiments, the antisense oligonucleotides are administered to the subject once per day, once per week, twice per week, once per month, twice per month, three times per month, or on any other regular schedule.

In some embodiments, the methods of the disclosure involve reduction of the expression of complement C5 (e.g., complement C5 mRNA and/or complement C5 protein) in the cell or subject (e.g., in cells of the subject) following administration of the antisense oligonucleotide. In some embodiments, the method causes reduction of expression of complement C5 by at least 5%, 10%, 25%, 40%, 50%, 75%, or more, relative to a control (e.g., cell or subject prior to administration of the oligonucleotide). In some embodiments, the methods result in reduced expression of complement C5 (e.g., complement C5 mRNA and/or complement C5 protein) by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to translation of the pre-mRNA prior to administration.

In some embodiments, the methods of the disclosure involve reduction of the translation of complement C5 mRNA in the cell or subject (e.g., in cells of the subject) following administration of the antisense oligonucleotide. In some embodiments, the method causes reduction of translation of complement C5 mRNA by at least 5%, 10%, 25%, 40%, 50%, 75%, or more, relative to a control (e.g., cell or subject prior to administration of the oligonucleotide). In some embodiments, the methods result in reduced translation of complement C5 mRNA in the cell or subject (e.g., in cells of the subject) by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to translation of the pre-mRNA prior to administration.

In some embodiments, the methods of the disclosure involve reduction of the processing of complement C5 pre-mRNA in the cell or subject (e.g., in cells of the subject) following administration of the antisense oligonucleotide. In some embodiments, the method causes reduction of processing of complement C5 pre-mRNA by at least 5%, 10%, 25%, 40%, 50%, 75%, or more, relative to a control (e.g., cell or subject prior to administration of the oligonucleotide). In some embodiments, the methods result in decreased translation of a complement C5 pre-mRNA by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to translation of the pre-mRNA prior to administration.

The efficacy of treatment resulting from the administration of the antisense oligonucleotides may be assessed using any suitable methods. In some embodiments, the efficacy of treatment may be assessed by evaluation of observation of symptoms associated with the disease or dysfunction.

### Kits

The disclosure further provides kits for use in a method of the disclosure. The antisense oligonucleotides described herein may, in some embodiments, be assembled into pharmaceutical or diagnostic or research kits to facilitate their methods of use. In some embodiments, a kit comprises a container housing an antisense oligonucleotide. In further embodiments, the kit comprises a container housing a pharmaceutically acceptable carrier and/or excipient. The antisense oligonucleotide may be housed in the same container as any carriers and/or excipients. Alternatively, the oligonucleotide may be housed in a first container and the carriers and/or excipients are housed in one or more different containers. The kits may further include instructions for use. In some embodiments, oligonucleotides in a kit may be in a pharmaceutical formulation and dosage suitable for a particular application and for a method of administration. Kits for research purposes may contain the components in appropriate concentrations or quantities for running various experiments.

Each of the compositions of the kit, where applicable, may be provided in liquid form (e.g., in solution), or in solid form, (e.g., a dry powder). As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and administering it to a subject. The kit may include a container housing agents described herein. The agents may be in the form of a liquid, gel or solid (powder). The agents may be prepared sterilely, packaged in syringe and shipped refrigerated. Alternatively it may be housed in a vial or other container for storage. A second container may have other agents prepared sterilely. Alternatively the kit may include the active agents premixed and shipped in a syringe, vial, tube, or other container. The kit may have one or more or all of the components required to administer the agents to an animal, such as a syringe, topical application devices, or intravenous needle tubing and bags.

### EXAMPLES

### Example 1—Targeting Complement C5 Pre-mRNA in HepG2 Cells

Antisense oligonucleotides (ASOs) provided in Table 2 were designed and synthesized. Specifically, the antisense oligonucleotides of this Example were gapmer antisense oligonucleotides comprising a central segment of 10 deoxyribonucleotides flanked by 5 ribonucleotides on either side of the central segment. The thymines shown in the ribonucleotide segments of SEQ ID NOs: 1-86 were replaced with uracils and each of the cytosines were 5-methyl cytosines. Furthermore, each of the RNA nucleotides were modified to be 2-O-methoxyethyl (2′-MOE) RNA nucleotides and each of the internucleotide linkages of each gapmer was a phosphorothioate linkage.

These gapmer antisense oligonucleotides (ASOs) were subsequently tested for their abilities to target a Complement C5 pre-mRNA (comprised with the reverse complement of NCBI NG_007364.1) for inhibition of Complement C5 gene expression in cellulo.

HepG2 cells were plated in 96-well tissue culture treated plates at a final volume of 100 μl/well (11,200 total cells/well). After two days of cell growth and recovery, the cell culture media (EMEM (ATCC #30-2003) and 10% heat inactivated fetal bovine serum) was removed and replaced with 100 μl of fresh, warmed medium. Cells were dosed with individual gapmer ASOs using lipofectamine transfection reagent at varying concentrations of gapmer ASO in OptiMEM (ThermoFisher #31985070). Negative controls experiments using buffer only (i.e., no ASO) were also performed. After 48 hours of ASO treatment, the cell media was removed and the cells were lysed. Primary screening was performed using 200 nM oligonucleotide. Dose-response experiments were performed using 1 nM, 3 nM, 10 nM, 30 nM, 100 nM and 300 nM oligonucleotide.

Levels of secreted Complement C5 protein were analyzed using an enzyme-linked immunosorbent assay (ELISA) after 24 hours and 48 hours, 72 hours and 96 hours of ASO treatment. Levels of Complement C5 gene expression were analyzed after 48 hours of ASO treatment using quantitative PCR (qPCR). The qPCR experiment analyzed gene expression using the following probes: Hs01004345_g1 FAM (genomic C5); Hs01004349_m1 FAM (mature C5); and Hs_99999903_m1 VIC (Actin B). Actin B was used as an endogenous control gene.

The ability of each individual gapmer ASO to inhibit gene expression of Complement C5 was assessed by determining its half maximal effective concentration (EC-50) across the range of tested concentrations. See, Table 2. Several gapmer ASOs had EC-50 values of greater than 50 nM in HepG2 cells. Note that having relatively high EC-50 values in HepG2 cells does not necessarily indicate that these gapmer ASOs would lack efficacy in other cell lines or in vivo. Gapmer ASOs having a sequence as set forth in any one of SEQ ID NOs: 4, 11, 12, 34, 39, 47, 53, 55, 56, 57, 60, 62, 66, 69, 71, 77, 86, 99, 105, 108, 109, 110, 115, and 119 had EC-50 values of less than 50 nM for inhibition of Complement C5 gene expression in HepG2 cells. These data demonstrate that targeting intronic regions of Complement C5 pre-mRNA using antisense oligonucleotides are effective in targeting Complement C5 pre-mRNA to inhibit gene expression.

TABLE 2
Antisense oligonucleotides for targeting Complement C5 pre-mRNA

| | | | SEQ ID | Intron of | |
|---|---|---|---|---|---|
| | SEQ | | NO of | Complement | |
| Antisense | ID NO | | Target | C5 gene | EC-50 |
| Oligonucleotide (ASO)* | of ASO | Target Sequence | Sequence | (NG_007364.1) | (nM) |
| gctggtctatgtgatggtag | 1 | ctaccatcacatagaccagc | 151 | Intron 1 | >100 |
| atcagaggctgtatcagcac | 2 | gtgctgatacagcctctgat | 152 | Intron 1 | >100 |
| cctctaacagaagaaccatg | 3 | catggttcttctgttagagg | 153 | Intron 3 | >100 |
| tggttcattgggaataggtc | 4 | gacctattcccaatgaacca | 154 | Intron 3 | 19.95 |
| caacctttgtgggcagagac | 5 | gtctctgcccacaaaggttg | 155 | Intron 3 | >100 |
| ggtcatacagctcacagcag | 6 | ctgctgtgagctgtatgacc | 156 | Intron 3 | >100 |
| gcccaagtcagaaagtcaca | 7 | tgtgactttctgacttgggc | 157 | Intron 3 | >100 |
| agtgtagtgtgaggacaggg | 8 | ccctgtcctcacactacact | 158 | Intron 4 | >100 |
| ttggactacacatgggggtc | 9 | gacccccatgtgtagtccaa | 159 | Intron 4 | >100 |
| gcaaggaacaagaccacacc | 10 | ggtgtggtcttgttccttgc | 160 | Intron 5 | >100 |
| gcttatgaagagccaggtga | 11 | tcacctggctcttcataagc | 161 | Intron 5 | 30.1 |
| gctcttcaggggactagatc | 12 | gatctagtcccctgaagagc | 162 | Intron 6 | 18.31 |
| ggtatagttggacaagctgt | 13 | acagcttgtccaactatacc | 163 | Intron 6 | >100 |
| agacatatggacaggacctt | 14 | aaggtcctgtccatatgtct | 164 | Intron 7 | >100 |
| ggcagctaacctagtgtaga | 15 | tctacactaggttagctgcc | 165 | Intron 8 | >100 |
| tttcaccaacaggcttcatc | 16 | gatgaagcctgttggtgaaa | 166 | Intron 12 | >100 |
| caacaccctaccctaaacct | 17 | aggtttagggtagggtgttg | 167 | Intron 14 | >100 |
| ggaatctcaagggatacctg | 18 | caggtatcccttgagattcc | 168 | Intron 16 | >100 |
| ggttaacttgaggggcagtc | 19 | gactgcccctcaagttaacc | 169 | Intron 17 | >100 |
| tagggcagggagaggaatac | 20 | gtattcctctccctgcccta | 170 | Intron 17 | >100 |
| gcagtggccagaaggtttac | 21 | gtaaaccttctggccactgc | 171 | Intron 18 | >100 |
| gtagccaagatcagtgtgag | 22 | ctcacactgatcttggctac | 172 | Intron 20 | >100 |
| gtggaagcaaaatatttaaa | 23 | tttaaatattttgcttccac | 173 | Intron 5 | >100 |
| cagaaactatagaacaggct | 24 | agcctgttctatagtttctg | 174 | Intron 2 | >100 |
| tctctggacccttagtatta | 25 | taatactaagggtccagaga | 175 | Intron 2 | >100 |
| gcaattgaaagtctctttgg | 26 | ccaaagagactttcaattgc | 176 | Intron 2 | >100 |
| gaattgagagtcttttgagc | 27 | gctcaaaagactctcaattc | 177 | Intron 3 | >100 |
| tggacaaagactgatgaccc | 28 | gggtcatcagtctttgtcca | 178 | Intron 3 | >100 |
| agggtcatacagctcacagc | 29 | gctgtgagctgtatgaccct | 179 | Intron 3 | >100 |
| gcttggctctatcttaagtg | 30 | cacttaagatagagccaagc | 180 | Intron 3 | >100 |
| gtagtaggtgatgaagctgg | 31 | ccagcttcatcacctactac | 181 | Intron 3 | >100 |
| gaggtcagtttggcttgtgt | 32 | acacaagccaaactgacctc | 182 | Intron 3 | >100 |
| gttaacaaggcaaaggaggg | 33 | ccctcctttgccttgttaac | 183 | Intron 3 | >100 |
| ggctagtttttaacccagtt | 34 | aactgggttaaaaactagcc | 184 | Intron 3 | 11.4 |
| ggcaccataaacccagtatc | 35 | gatactgggtttatggtgcc | 185 | Intron 3 | >100 |
| taactcacagctgtttgaga | 36 | tctcaaacagctgtgagtta | 186 | Intron 3 | >100 |
| ttgggagcttgattcagaca | 37 | tgtctgaatcaagctcccaa | 187 | Intron 3 | 73.81 |
| gatttagtgtagtgtgagga | 38 | tcctcacactacactaaatc | 188 | Intron 4 | >100 |
| tcttagtgattggtgggtgg | 39 | ccacccaccaatcactaaga | 189 | Intron 4 | 35.89 |
| ggaccattagtagtatacct | 40 | aggtatactactaatggtcc | 190 | Intron 4 | >100 |
| tctgagctcatggtatttgt | 41 | acaaataccatgagctcaga | 191 | Intron 4 | >100 |
| ctcacctctcattggtcttc | 42 | gaagaccaatgagaggtgag | 192 | Intron 4 | >100 |
| tacatggatggtcttcacta | 43 | tagtgaagaccatccatgta | 193 | Intron 4 | >100 |
| agggcatttgagctgacatt | 44 | aatgtcagctcaaatgccct | 194 | Intron 4 | >100 |
| catagcaggcaacataagtg | 45 | cacttatgttgcctgctatg | 195 | Intron 4 | >100 |
| ttctttggactacacatggg | 46 | cccatgtgtagtccaaagaa | 196 | Intron 4 | >100 |
| tggccccctttaactggtta | 47 | taaccagttaaagggggcca | 197 | Intron 5 | 20.81 |
| acaagaccacaccagccttt | 48 | aaaggctggtgtggtcttgt | 198 | Intron 5 | >100 |
| agcaaccagagcaaggaaca | 49 | tgttccttgctctggttgct | 199 | Intron 5 | >100 |
| gttggagaaatgatagcagt | 50 | actgctatcatttctccaac | 200 | Intron 5 | >100 |
| gagccaggtgaagcccattc | 51 | gaatgggcttcacctggctc | 201 | Intron 5 | 54.64 |
| agcttctactctgggtcata | 52 | tatgacccagagtagaagct | 202 | Intron 5 | 66.33 |
| gggcctattatgttgaactg | 53 | cagttcaacataataggccc | 203 | Intron 5 | 21.56 |
| ggttggaaacaaagcatggt | 54 | accatgctttgtttccaacc | 204 | Intron 5 | >100 |
| gccagtacttctagccatac | 55 | gtatggctagaagtactggc | 205 | Intron 6 | 39.05 |
| ctagtcaagctcttcagggg | 56 | cccctgaagagcttgactag | 206 | Intron 6 | 34.45 |
| ggggtaaggaaacctaattg | 57 | caattaggtttccttacccc | 207 | Intron 6 | 27.75 |
| gcctaggctcagggggactt | 58 | aagtccccctgagcctaggc | 208 | Intron 7 | >100 |
| ggagtgattaatacaaaggg | 59 | ccctttgtattaatcactcc | 209 | Intron 8 | >100 |
| aacctagtgtagaagagtca | 60 | tgactcttctacactaggtt | 210 | Intron 8 | 37.99 |
| caacccctcagcattattga | 61 | tcaataatgctgaggggttg | 211 | Intron 8 | >100 |
| tgtctctgggcatataaggg | 62 | cccttatatgcccagagaca | 212 | Intron 8 | 15.74 |
| gattaagcttcaccatctac | 63 | gtagatggtgaagcttaatc | 213 | Intron 9 | >100 |
| gtgttcacagagcatagata | 64 | tatctatgctctgtgaacac | 214 | Intron 9 | >100 |
| gaaggttttgagtactacac | 65 | gtgtagtactcaaaaccttc | 215 | Intron 15 | >100 |
| taggttaacttgaggggcag | 66 | ctgcccctcaagttaaccta | 216 | Intron 17 | 38.22 |
| gcctgatgattttgaagact | 67 | agtcttcaaaatcatcaggc | 217 | Intron 17 | >100 |
| cagaaggtttactagaggac | 68 | gtcctctagtaaaccttctg | 218 | Intron 18 | >100 |
| ggccaagtaaaatttgtgtg | 69 | cacacaaattttacttggcc | 219 | Intron 20 | 15.7 |
| gtggcatgattcataggatt | 70 | aatcctatgaatcatgccac | 220 | Intron 20 | 77.02 |
| ggtctgactgtcttagtatc | 71 | gatactaagacagtcagacc | 221 | Intron 20 | 30.39 |
| acagttgagcagagtttggc | 72 | gccaaactctgctcaactgt | 222 | Intron 20 | 51.96 |
| gagtgatcttagaaatacta | 73 | tagtatttctaagatcactc | 223 | Intron 1 | |
| gttatttgatcaaggttgtt | 74 | aacaaccttgatcaaataac | 224 | Intron 2 | |
| tgtctctaagcaatctatac | 75 | gtatagattgcttagagaca | 225 | Intron 2 | |
| gggatgaaagttaattacaa | 76 | ttgtaattaactttcatccc | 226 | Intron 2 | |
| catatatttctgtgatctag | 77 | ctagatcacagaaatatatg | 227 | Intron 3 | 39.5 |
| tacatcatcttacagtaaag | 78 | ctttactgtaagatgatgta | 228 | Intron 3 | |
| ttgggcaatattctaaagtc | 79 | gactttagaatattgcccaa | 229 | Intron 3 | |
| ttacacaggccttaatatgg | 80 | ccatattaaggcctgtgtaa | 230 | Intron 3 | |
| aaggaggggcaagaatgtac | 81 | gtacattcttgcccctcctt | 231 | Intron 3 | |
| gtaaaattctagggtgtgaa | 82 | ttcacaccctagaattttac | 232 | Intron 3 | |
| gacatggacatcttttaatg | 83 | cattaaaagatgtccatgtc | 233 | Intron 3 | |
| aaaagatatggcttattggc | 84 | gccaataagccatatctttt | 234 | Intron 3 | |
| gcatagcctgacacatacta | 85 | tagtatgtgtcaggctatgc | 235 | Intron 3 | |
| ttacatctgtaattaggcca | 86 | tggcctaattacagatgtaa | 236 | Intron 3 | 21.5 |
| tagtggatttaaagtacagt | 87 | actgtactttaaatccacta | 237 | Intron 3 | |
| ttgtatgagcctttcacata | 88 | tatgtgaaaggctcatacaa | 238 | Intron 4 | |
| gcaagattgtacttaaatgg | 89 | ccatttaagtacaatcttgc | 239 | Intron 4 | |
| gtctgttacatatgtcatgt | 90 | acatgacatatgtaacagac | 240 | Intron 4 | |
| ctgatagaaaaagggcattt | 91 | aaatgccctttttctatcag | 241 | Intron 4 | |
| acaagaattatacttgtagc | 92 | gctacaagtataattcttgt | 242 | Intron 4 | |
| ccatgactgaaattcacagt | 93 | actgtgaatttcagtcatgg | 243 | Intron 4 | |
| aatatggttcatttcagcca | 94 | tggctgaaatgaaccatatt | 244 | Intron 4 | |
| tgagatagggagtaaaacaa | 95 | ttgttttactccctatctca | 245 | Intron 5 | |
| acattgagataagtgtcatg | 96 | catgacacttatctcaatgt | 246 | Intron 5 | |
| ttataagctagtggggaata | 97 | tattccccactagcttataa | 247 | Intron 5 | |
| atacaagacaagcttataca | 98 | tgtataagcttgtcttgtat | 248 | Intron 5 | |
| tgaactcatatcatctgtgg | 99 | ccacagatgatatgagttca | 249 | Intron 8 | 32.2 |
| gcttagagctcatatggaat | 100 | attccatatgagctctaagc | 250 | Intron 9 | |
| ataggaacttatgagaattg | 101 | caattctcataagttcctat | 251 | Intron 14 | >100 |
| gttaaatcaagaactaagtc | 102 | gacttagttcttgatttaac | 252 | Intron 14 | |
| aggtactaattgttacatgg | 103 | ccatgtaacaattagtacct | 253 | Intron 14 | |
| acttgtaaatagatctagtc | 104 | gactagatctatttacaagt | 254 | Intron 15 | |
| ttaactctggcctagtgtac | 105 | gtacactaggccagagttaa | 255 | Intron 17 | 38.18 |
| cctgttggtacactaaattt | 106 | aaatttagtgtaccaacagg | 256 | Intron 17 | |
| ccagatgattatagttgaaa | 107 | tttcaactataatcatctgg | 257 | Intron 17 | |
| tatggccattaatctgtaac | 108 | gttacagattaatggccata | 258 | Intron 17 | 27.9 |
| ctagctaatgggtaatgtca | 109 | tgacattacccattagctag | 259 | Intron 18 | 28.8 |
| acagctattgtctaccaact | 110 | agttggtagacaatagctgt | 260 | Intron 18 | 36.7 |
| atattctaaaatagggaagc | 111 | gcttccctattttagaatat | 261 | Intron 18 | >100 |
| ctctatgatgagaattcaag | 112 | cttgaattctcatcatagag | 262 | Intron 18 | |
| acccaacttaacagagacat | 113 | atgtctctgttaagttgggt | 263 | Intron 18 | |
| gaatattgattaacccaact | 114 | agttgggttaatcaatattc | 264 | Intron 18 | |
| aatctggttaatgagttagg | 115 | cctaactcattaaccagatt | 265 | Intron 20 | 40.4 |
| cataattttagtcagtgtgg | 116 | ccacactgactaaaattatg | 266 | Intron 20 | 57.5 |
| ttatcaatgaggactgattt | 117 | aaatcagtcctcattgataa | 267 | Intron 20 | |
| caaaccctgtgagtaaattc | 118 | gaatttactcacagggtttg | 268 | Intron 20 | |
| gtctgttttgatgagatggt | 119 | accatctcatcaaaacagac | 269 | Intron 20 | 27.53 |
| tatatgtctaagactaggtg | 120 | cacctagtottagacatata | 270 | Intron 20 | |

*ASO sequences are 5-10-5 configuration gapmers comprising 5 RNA nucleotides on both sides
of a central 10 DNA nucleotide region. Accordingly, each ‘t’ or thymine contained within the
RNA portions of the gapmers may be replaced with ‘u’ or uracil.

### Example 2—Targeting Regions of Interest within Complement C5 Pre-mRNA

Certain antisense oligonucleotides (ASOs) identified in Example 1 were selected for further analysis. Specifically, antisense oligonucleotides having the nucleic acid sequences of SEQ ID NOs: 34, 62, and 69 (which demonstrated EC-50 values of 11.4 nM, 15.74 nM, and 15.7 nM, respectively) were subjected to tilling experiments to demonstrate the ability of targeting Complement C5 sequences that overlapped with the corresponding target sequences complementary to having the nucleic acid sequences of SEQ ID NOs: 34, 62, and 69 (SEQ ID NOs: 184, 212, and 219, respectively). Antisense oligonucleotides sharing 11, 13, 15, or 17 contiguous nucleotides with the antisense oligonucleotides having the nucleic acid sequences of SEQ ID NOs: 34, 62, and 69 were designed and tested for their abilities to target a Complement C5 pre-mRNA (comprised with the reverse complement of NCBI NG_007364.1) for inhibition of Complement C5 gene expression in cellulo as performed in Example 1. Designed sequences were placed into the same gapmer framework as described in Example 1.

For example, the nucleic acid sequence of SEQ ID NO: 121 comprised the 11 contiguous nucleotides at the 3′ end of SEQ ID NO: 34; the nucleic acid sequence of SEQ ID NO: 122 comprised the 13 contiguous nucleotides at the 3′ end of SEQ ID NO: 34; the nucleic acid sequence of SEQ ID NO: 123 comprised the 15 contiguous nucleotides at the 3′ end of SEQ ID NO: 34; and the nucleic acid sequence of SEQ ID NO: 124 comprised the 17 contiguous nucleotides at the 3′ end of SEQ ID NO: 34. The nucleic acid sequence of SEQ ID NO: 128 comprised the 11 contiguous nucleotides at the 5′ end of SEQ ID NO: 34; the nucleic acid sequence of SEQ ID NO: 127 comprised the 13 contiguous nucleotides at the 5′ end of SEQ ID NO: 34; the nucleic acid sequence of SEQ ID NO: 126 comprised the 15 contiguous nucleotides at the 5′ end of SEQ ID NO: 34; and the nucleic acid sequence of SEQ ID NO: 125 comprised the 17 contiguous nucleotides at the 5′ end of SEQ ID NO: 34. Sequences sharing sequence identity with the nucleic acid sequences of SEQ ID NO: 62 and 69 were similarly designed.

The ability of each individual gapmer ASO to inhibit gene expression of Complement C5 was assessed by determining its half maximal effective concentration (EC-50) across the range of tested concentrations. Sec, Table 3. Several gapmer ASOs had EC-50 values of greater than 50 nM in HepG2 cells. Note that having relatively high EC-50 values in HepG2 cells does not necessarily indicate that these gapmer ASOs would lack efficacy in other cell lines or in vivo. These data demonstrate that targeting certain intronic regions of Complement C5 pre-mRNA can be performed using antisense oligonucleotides having at least 11, 13, 15, or 17 contiguous nucleotides with an identified nucleic acid sequence.

TABLE 3
Antisense oligonucleotides for targeting Complement C5 pre-mRNA

| | | | | Intron of | |
|---|---|---|---|---|---|
| | SEQ | | SEQ ID NO | Complement | |
| Antisense | ID NO | | of Target | C5 gene | EC-50 |
| Oligonucleotide (ASO)* | of ASO | Target Sequence | Sequence | (NG_007364.1) | (nM) |
| ggctagtttttaacccagtt | 34 | aactgggttaaaaactagcc | 184 | Intron 3 | 11.4 |
| ttaacccagtttgtgtttag | 121 | ctaaacacaaactgggttaa | 271 | Intron 3 | >100 |
| ttttaacccagtttgtgttt | 122 | aaacacaaactgggttaaaa | 272 | Intron 3 | >100 |
| gtttttaacccagtttgtgt | 123 | acacaaactgggttaaaaac | 273 | Intron 3 | >100 |
| tagtttttaacccagtttgt | 124 | acaaactgggttaaaaacta | 274 | Intron 3 | >100 |
| ttggctagtttttaacccag | 125 | tgggttaaaaactagccaaa | 275 | Intron 3 | 32 |
| tatttggctagtttttaacc | 126 | ggttaaaaactagccaaata | 276 | Intron 3 | >100 |
| ctatttggctagtttttaac | 127 | gttaaaaactagccaaatag | 277 | Intron 3 | >100 |
| tactatttggctagttttta | 128 | taaaaactagccaaatagta | 278 | Intron 3 | >100 |
| tgtctctgggcatataaggg | 62 | cccttatatgcccagagaca | 212 | Intron 8 | 15.74 |
| ataagggaaatgattcagac | 129 | gtctgaatcatttcccttat | 279 | Intron 8 | >100 |
| gcatataagggaaatgattc | 130 | gaatcatttcccttatatgc | 280 | Intron 8 | >100 |
| gggcatataagggaaatgat | 131 | atcatttcccttatatgccc | 281 | Intron 8 | 50 |
| ctgggcatataagggaaatg | 132 | catttcccttatatgcccag | 282 | Intron 8 | >100 |
| tctctgggcatataagggaa | 133 | ttcccttatatgcccagaga | 283 | Intron 8 | 13.7 |
| gctgtctctgggcatataag | 134 | cttatatgcccagagacagc | 284 | Intron 8 | 15.4 |
| tatgctgtctctgggcatat | 135 | atatgcccagagacagcata | 285 | Intron 8 | 32.6 |
| actatgctgtctctgggcat | 136 | atgcccagagacagcatagt | 286 | Intron 8 | >100 |
| ggccaagtaaaatttgtgtg | 69 | cacacaaattttacttggcc | 219 | Intron 20 | 15.7 |
| aagtaaaatttgtgtgtgca | 137 | tgcacacacaaattttactt | 287 | Intron 20 | 36.4 |
| ccaagtaaaatttgtgtgtg | 138 | cacacacaaattttacttgg | 288 | Intron 20 | >100 |
| gtggccaagtaaaatttgtg | 139 | cacaaattttacttggccac | 289 | Intron 20 | 20.6 |
| cctgtggccaagtaaaattt | 140 | aaattttacttggccacagg | 290 | Intron 20 | >100 |
| catcctgtggccaagtaaaa | 141 | ttttacttggccacaggatg | 291 | Intron 20 | >100 |
| ttacatcctgtggccaagta | 142 | tacttggccacaggatgtaa | 292 | Intron 20 | 48.7 |
| atttacatcctgtggccaag | 143 | cttggccacaggatgtaaat | 293 | Intron 20 | 29.33 |
| cccatttacatcctgtggcc | 144 | ggccacaggatgtaaatggg | 294 | Intron 20 | >100 |

*ASO sequences are 5-10-5 configuration gapmers comprising 5 RNA nucleotides on both sides
of a central 10 DNA nucleotide region. Accordingly, each ‘t’ or thymine contained within the
RNA portions of the gapmers may be replaced with ‘u’ or uracil.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or.” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of.” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either.” “one of.” “only one of.” or “exactly one of.” “Consisting essentially of.” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B.” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

What is claimed is:

1. An antisense oligonucleotide comprising a sequence that is complementary to an intron of a complement C5 pre-mRNA.

2. The antisense oligonucleotide of claim 1, wherein the intron is intron 1, 2, 3, 4, 5, 6, 7, 8, 9, 12, 14, 15, 16, 17, 18, or 20.

3. The antisense oligonucleotide of claim 1 or 2, wherein the oligonucleotide comprises a length of at least 8, 10, 15, or 20 nucleotides.

4. The antisense oligonucleotide of any one of claims 1-3, wherein the sequence that is complementary to the intron has a length of at least 8, 10, 15, or 20 nucleotides.

5. The antisense oligonucleotide of any one of claims 1-4, wherein the oligonucleotide has a length of 8-100 nucleotides, optionally a length of 18-30 nucleotides.

6. The antisense oligonucleotide of any preceding claim, wherein the oligonucleotide binds to the intron and selectively inhibits translation of the complement C5 pre-mRNA.

7. The antisense oligonucleotide of any preceding claim, wherein the oligonucleotide binds to the intron and selectively inhibits downstream expression of complement C5 protein.

8. The antisense oligonucleotide of any preceding claim, wherein the oligonucleotide is complementary to an intron of a complement C5 pre-mRNA comprising the nucleic acid sequence of SEQ ID NO: 301.

9. The antisense oligonucleotide of any preceding claim, further comprising a second sequence is complementary to an exon of the complement C5 pre-mRNA.

10. The antisense oligonucleotide of any one of claims 1-8, wherein the entire length of the oligonucleotide is complementary to a segment of a complement C5 pre-mRNA, optionally an intron of a complement C5 pre-mRNA.

11. The antisense oligonucleotide of any preceding claim, wherein the oligonucleotide comprises at least one modified internucleotide linkage.

12. The antisense oligonucleotide of claim 11, wherein the at least one modified internucleotide linkage is a phosphorothioate linkage.

13. The antisense oligonucleotide of claim 12, wherein the majority of internucleotide linkages of the oligonucleotide are phosphorothioate linkages.

14. The antisense oligonucleotide of claim 12, wherein each of internucleotide linkages of the oligonucleotide is a phosphorothioate linkage.

15. The antisense oligonucleotide of any preceding claim, wherein the oligonucleotide comprises at least one modified nucleotide.

16. The antisense oligonucleotide of claim 15, wherein the majority of nucleotides of the oligonucleotide are modified nucleotides.

17. The antisense oligonucleotide of claim 15, wherein each of the nucleotides of the oligonucleotide is a modified nucleotide.

18. The antisense oligonucleotide of any one of claims 15-17, wherein the at least one modified nucleotide is a 2′-modified nucleotide.

19. The antisense oligonucleotide of claim 18, wherein the 2′-modified nucleotide is a 2′-O-methyl nucleotide, 2′-fluoro (2′-F) nucleotide, or 2′-O-methoxyethyl (2′-MOE) nucleotide.

20. The antisense oligonucleotide of claim 19, wherein each of the nucleotides of the oligonucleotide is a 2′-O-methoxyethyl (2′-MOE) nucleotide.

21. The antisense oligonucleotide of any preceding claim, wherein each of internucleotide linkages of the oligonucleotide is a phosphorothioate linkage, and wherein each of the nucleotides of the oligonucleotide is a 2′-O-methoxyethyl (2′-MOE) nucleotide.

22. The antisense oligonucleotide of any preceding claim, wherein the oligonucleotide is a gapmer.

23. The antisense oligonucleotide of claim 22, wherein the gapmer comprises a central segment of 5-20 deoxyribonucleotides flanked by 2-15 ribonucleotides on either side of the central segment.

24. The antisense oligonucleotide of claim 22 or 23, wherein the gapmer comprises a central segment of 10 deoxyribonucleotides flanked by 5 ribonucleotides on either side of the central segment.

25. The antisense oligonucleotide of claim 24, wherein each of internucleotide linkages of the oligonucleotide is a phosphorothioate linkage, and wherein each of the ribonucleotides of the oligonucleotide is a 2′-O-methoxyethyl (2′-MOE) ribonucleotide.

26. The antisense oligonucleotide of any preceding claim, wherein the oligonucleotide is single-stranded or double-stranded.

27. The antisense oligonucleotide of any preceding claim, wherein the oligonucleotide comprises at least 10, at least 11, at least 12, at least 13, at least 14, at least 15, at least 16, at least 17, at least 18, at least 19, or at least 20 contiguous nucleotides of a nucleic acid sequence as set forth in any one of SEQ ID NOs: 1-150, optionally wherein one or more of the thymine nucleobases of the nucleic acid sequence are replaced with uracil nucleobases.

28. The antisense oligonucleotide of any preceding claim, wherein the oligonucleotide is a gapmer comprising a central segment of 10 deoxyribonucleotides flanked by 5 ribonucleotides on either side of the central segment, wherein the oligonucleotide comprises a nucleic acid sequence that as set forth in any one of SEQ ID NOs: 1-150, wherein the thymines in the ribonucleotide segments are replaced with uracils.

29. The antisense oligonucleotide of any preceding claim, wherein at least one of the cytosines in the oligonucleotide is a 5-methylcytosine.

30. The antisense oligonucleotide of any preceding claim, wherein each of the cytosines in the oligonucleotide is a 5-methylcytosine.

31. The antisense oligonucleotide of any preceding claim, wherein the oligonucleotide is complementary to at least 10, at least 11, at least 12, at least 13, at least 14, at least 15, at least 16, at least 17, at least 18, at least 19, or at least 20 contiguous nucleotides of a nucleic acid sequence that as set forth in any one of SEQ ID NOs: 151-300.

32. A pharmaceutical composition comprising (i) the antisense oligonucleotide of any one of claims 1-31; and (ii) a pharmaceutically acceptable carrier and/or excipient.

33. The pharmaceutical composition of claim 32, wherein the pharmaceutically acceptable carrier is a solvent, aqueous buffer, liposome, polymeric micelle, nucleic acid nanostructure or nanoparticle.

34. The pharmaceutical composition of claim 32 or 33, wherein the excipient is an antiadherent, binder, coating, or preservative.

35. A kit comprising a container housing the antisense oligonucleotide of any one of claims 1-31.

36. The kit of claim 35, wherein the kit further comprises a container housing a pharmaceutically acceptable carrier and/or excipient.

37. The kit of claim 36, wherein the antisense oligonucleotide and the pharmaceutically acceptable carrier and/or excipient are housed in the same container.

38. A method of inhibiting expression of complement C5 in a cell, the method comprising contacting the cell with the antisense oligonucleotide of any one of claims 1-31 or the pharmaceutical composition of any one of claims 32-34.

39. A method of inhibiting translation of a complement C5 pre-mRNA in a cell, the method comprising contacting the cell with the antisense oligonucleotide of any one of claims 1-31 or the pharmaceutical composition of any one of claims 32-34.

40. The method of claim 39, wherein the complement C5 pre-mRNA comprises the nucleic acid sequence of SEQ ID NO: 301.

41. The method of any one of claims 38-40, wherein the method reduces expression of complement C5 by at least 5%, 10%, 25%, 40%, 50%, 75%, or more, relative to a control.

42. The method of claim 39 or 40, wherein the method reduces translation of complement C5 pre-mRNA by at least 5%, 10%, 25%, 40%, 50%, 75%, or more, relative to a control.

43. The method of any one of claims 38-42, wherein the cell is a mammalian cell, optionally a human cell.

44. The method of any one of claims 38-43, wherein the cell is from a human subject having or suspected of having a disease or disorder associated with complement C5 dysfunction.

45. The method of claim 44, wherein the disease or disorder associated with complement C5 dysfunction is a neurological disease, a primary dysregulation disorder, an autoimmune disease, an inflammatory disease, a degenerative disease, an ischaemia-reperfusion, or an acute injury

46. The method of claim 44, wherein the disease or disorder associated with complement C5 dysfunction is multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD)

47. The method of claim 44, wherein the disease or disorder associated with complement C5 dysfunction is paroxysmal nocturnal hemoglobinuria (PNH) or atypical hemolytic uremic syndrome.

48. A method of treating a disease or disorder associated with complement C5 dysfunction of complement C5 in a subject, the method comprising administering an effective amount of the antisense oligonucleotide of any one of claims 1-31 or the pharmaceutical composition of any one of claims 32-34 to the subject, thereby treating the subject.

49. The method of claim 48, wherein the disease or disorder associated with complement C5 dysfunction is a neurological disease, a primary dysregulation disorder, an autoimmune disease, an inflammatory disease, a degenerative disease, an ischaemia-reperfusion, or an acute injury

50. The method of claim 48, wherein the disease or disorder associated with complement C5 dysfunction is multiple sclerosis (MS), neuromyelitis optica (NMO), neurotrauma, stroke, amyotrophic lateral sclerosis (ALS), Alzheimer's disease (AD), Parkinson's disease (PD), or Huntington's disease (HD)

51. The method of claim 48, wherein the disease or disorder associated with complement C5 dysfunction is paroxysmal nocturnal hemoglobinuria (PNH) or atypical hemolytic uremic syndrome.

52. The method of any one of claims 48-51, wherein the subject is a human subject.

53. The method of any one of claims 48-52, wherein the subject has elevated levels of complement C5 expression, relative to a healthy subject.

54. The method of any one of claims 48-53, wherein the antisense oligonucleotide or the pharmaceutical composition is administered by intrathecal injection, intravenous injection, intramuscular injection, inhalation, subcutaneous injection, and/or intracranial injection.

55. The method of any one of claims 48-54, wherein administration of the antisense oligonucleotide or the pharmaceutical composition results in decreased translation of a complement C5 pre-mRNA by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to translation of the pre-mRNA prior to administration.

56. The method of claim 55, wherein the complement C5 pre-mRNA comprises the nucleic acid sequence of SEQ ID NO: 301.

57. The method of any one of claims 48-56, wherein administration of the antisense oligonucleotide or the pharmaceutical composition results in decreased expression of complement C5 by at least 2-fold, at least 10-fold, at least 20-fold, at least 30-fold, at least 40-fold, at least 50-fold, at least 60-fold, at least 70-fold, at least 80-fold, at least 90-fold, or at least 100-fold relative to expression of complement C5 prior to administration.
